SEQUENCE LISTING (29 sequences)

SEQ ID NO: 1 - DNA, 1860 bp, Nicotiana tabacum
ccacgcgttcgggataacatcattatccttctctcctcttcttccttctttcaaccacaattctcactcccctctttcgtctctcttctccaacttcaatcccattttcaggcaaaaagctgtcatggcttcaatttcagcagcttctgccacagctacagcttctacaaagcttgcatacccttattccccttcttcctcaagcagcagcagcaacactgctgctgtattcccttcaaattcctcaaagcttatcctttcctcttcttttacacccaccccttcaacccttttcctccactcaccaacaactactccttccaccacccacccccgtcggttcactgtccgcgctgcacgtggcaaattcgagcgtaaaaaacctcacgtcaacattggtacaattggccacgttgaccatggaaagaccacactcacagctgctttgaccatggcgcttgcctctatgggcaactccgcccccaagaaatatgacgaaattgatgctgcccctgaagaaagggcgcgtggtattactatcaacactgccactgtggaatatgaaacggaaaacagacattatgcacacgtggactgcccggggcatgctgattatgtcaagaacatgattactggtgctgcccaaatggatggggcaattcttgttgtgtcaggtgctgatggcccaatgccacagactaaagagcatattttgttagctaagcaagttggggtccctaatatggttgttttcttgaacaaacaagaccaagttgatgatgaggagttacttgagcttgttgagttggaggtaagagaattattgtcaagttatgagttccctggtgatgaaattcctattatttctggttctgcacttttagctttagaggctttgatggctaatcctagtattaaaaggggtgaaaatcaatgggttgataagatttatcaattgatggataatgttgatgaatatatccctatcccacaaagacaaactgaattgcctttcttgatggctattgaggatgttttctcgattaccggtagaggtactgtggcgacggggagagtagagagagggactgttaaggttggggaaattgttgatatagttggattgaaggatactaggaatactacagtgacaggggttgagatgtttcagaagattttggatgaagcgatggcgggagataatgtgggattgttgttgagaggtattcagaagattgatattcagagagggatggtgttggcgaagcccggaacaattactccgcacacaaagtttgaagctttggtgtatgtgttgaagaaggaagagggaggaaggcattccccgttctttgcgggttataggcctcaattttacatgaggacaactgatgtgactggaaaggttactgtgattatgagtgacaaaggagaggaatctaagatggtcatgcctggcgatcgtgtaaacatggtggttgagcttatcatgccggttgcatgtgagcaagggatgaggtttgctatcagggaaggaggaaagactgttggagctggtgttattcagaaaatcttagaatgatgaacttgcagctgagcatctcttttcacatgatcggcactttccattgaagttacttaatccattgtcatatatgcaacttcttggttacttttattatgtcttagaatcttactttagtagaagtatcctgttttaaacaccaaattctactgaacttttgggatttttcctcagtctcctctttcatttttcctttgcttgaaaggaatgagaacatttgatttcatgcactttatttaatttagaacaaatgtgcgactctgtttaaaattaag

SEQ ID NO: 2 - protein, 485 aa, Nicotiana tabacum
MASISAASATATASTKLAYPYSPSSSSSSSNTAAVFPSNSSKLILSSSFTPTPSTLFLHSPTTTPSTTHPRRFTVRAARGKFERKKPHVNIGTIGHVDHGKTTLTAALTMALASMGNSAPKKYDEIDAAPEERARGITINTATVEYETENRHYAHVDCPGHADYVKNMITGAAQMDGAILVVSGADGPMPQTKEHILLAKQVGVPNMVVFLNKQDQVDDEELLELVELEVRELLSSYEFPGDEIPIISGSALLALEALMANPSIKRGENQWVDKIYQLMDNVDEYIPIPQRQTELPFLMAIEDVFSITGRGTVATGRVERGTVKVGEIVDIVGLKDTRNTTVTGVEMFQKILDEAMAGDNVGLLLRGIQKIDIQRGMVLAKPGTITPHTKFEALVYVLKKEEGGRHSPFFAGYRPQFYMRTTDVTGKVTVIMSDKGEESKMVMPGDRVNMVVELIMPVACEQGMRFAIREGGKTVGAGVIQKILE

SEQ ID NO: 3 - DNA, 2342 bp, Nicotiana tabacum
gaattcttataatttaacgtaaaccaaataattaaaaaatcgatcttcatattgcagaaaaacagggattttatatatgtagttttaaaaaaagaaaaagaaaaagaaaaaatgtttccctgcccctaattcccaagaggaaagttgttgccgcagcgtagtgttatccaaataggtagaaatggatagaaaaatccccccaaaatgagtatatgagcggccctattttttccttccaagggatataacactattatccccttctcctcattttctttcaaatcccaattctcactcgctctatccttaactctcttttctctcccaacttcaatctccggccatggcttcaatttcagcagccaccgccacctcctccactaagcttgtatcatccaactccaccaatcctcttcttccttcctccactaaaccctctaagcttatcctatcctcttcctttacccctaattgttccacccttttcctccactcacccgccaccccttcttctactgccacccatcgccaccgccggtttactgtccgcgctgcccgtggcaaattcgagcggaaaaaaccccatgtcaacatcggtactattggccatgttgaccacggaaagactactctcaccgctgctttaaccatggctcttgcttctatgggaaactccgcccctaagaaatacgacgaaattgatgccgccccagaagagcgtgctcgtggtattactattaacactgctactgttgagtacgagactgagaaccgtcactacgcccacgtggattgccctggtcacgctgattatgtcaagaatatgattaccggtgctgcccaaatggacggcgctattcttgtttgttcaggtgctgatggtcccatgccacagactaaggaacacattttgcttgctaagcaagtaggtgtccccaacatggttgtgttcttgaacaaacaagatcaggttgatgatgaggagctgcttcagcttgttgagttggaggtaagagagttattgtcaagttatgagtttcctggtgatgatattcctattatttctggctctgctcttttggctttagaggctttgatggctaatcctagtattaagagaggtgaaaatcaatgggttgataagatatatgagttgatggacgctgttgatagttatattcctattccagttaggcaaactgaattgcctttcttgatggctattgaagatgtgttttccattactggtagaggtactgtggcgacggggagggtagagagagggactgttaggattggagacactgttgatattgtaggtttgaaggacactaggagtactaccgtgacgggtgttgagatgtttcaaaagattttggatgaagcaatggctggagacaatgtggggttgttgttgagaggtattcaaaagattgatattcagagaggaatggtgttggcaaaacccggaacaattacccctcacaccaagtttgaagctattgtgtatgtgttgaagaaggaggagggtggtaggcattcccccttcttttcagggtacaggcctcagttttacatgaggactactgatgtgaccggaaaggttacttccattacgactgataaaggagaggaatctaagatggtcatgcctggtgatcgtgtgaacttggtggttgagctcattatgccggtggcttgtgagcaagggatgagatttgccatcagggaaggaggaaagactgttggagctggtgtcattcagaaaattatcgagtgatcaaccaatagcggagcaatttcccttcaaatggtctggtccctgtcacacagtatttttaatccattgtcatatatgcagctttttggttacttttttcttatcttttagaatcctagttcagttggagaaatcttgttataagtcaatcctagtggacctttgcgagtttgcttcagtttcgtgctttgttttttcatttgtttgcaaggatggagaacattttgtctcttgtgcatcttactttctgctagaacaaattcatattgtgtttgaaactcgagtttaacatgctaatgaaattaggtgcatttgatatttgttttgacgaattgatgctgctaggaaacacccaaaaaaaagattactgagatgttttctttaccagcatatgtcgtttaaaaactaaaattgatggacatgagctgaccctgtaaggaaaatattgtaacagaacctcataagacttaagtttagtgatgcttaattgttattctcaattctggctttaatccctgagtagaactatctgaaatgatcagttggttctgtcttggaattc

SEQ ID NO: 4 - protein, 478 aa, Nicotiana tabacum
MASISAATATSSTKLVSSNSTNPLLPSSTKPSKLILSSSFTPNCSTLFLHSPATPSSTATHRHRRFTVRAARGKFERKKPHVNIGTIGHVDHGKTTLTAALTMALASMGNSAPKKYDEIDAAPEERARGITINTATVEYETENRHYAHVDCPGHADYVKNMITGAAQMDGAILVCSGADGPMPQTKEHILLAKQVGVPNMVVFLNKQDQVDDEELLQLVELEVRELLSSYEFPGDDIPIISGSALLALEALMANPSIKRGENQWVDKIYELMDAVDSYIPIPVRQTELPFLMAIEDVFSITGRGTVATGRVERGTVRIGDTVDIVGLKDTRSTTVTGVEMFQKILDEAMAGDNVGLLLRGIQKIDIQRGMVLAKPGTITPHTKFEAIVYVLKKEEGGRHSPFFSGYRPQFYMRTTDVTGKVTSITTDKGEESKMVMPGDRVNLVVELIMPVACEQGMRFAIREGGKTVGAGVIQKIIE

SEQ ID NO: 5 - DNA, 1758 bp, Arabidopsis thaliana
atccccaaatcttctccttctccaaaaaaatttcaaaccctagcttctcgatttctctcctctgctctccaattccatcttcccatggcgatttcggctccagccgcttgttcttcctcctctagaattctctgttcctactcctctccttctccttctctctgtcccgccatttccacctctggtaaactcaaaaccctaactctctcttcctccttcctcccttcttactcattaaccaccacctccgcttctcaatccactcgtcgctccttcaccgtccgcgccgctcgtggaaagttcgagaggaagaagcctcatgtcaacatcggaaccatcggtcatgttgaccatgggaaaactactttaaccgcagctctaaccatggctctcgcttccattggttccagcgtcgctaaaaagtacgacgagattgacgctgcgccggaggagagagctcgtggtatcacaatcaacactgctactgttgagtacgagactgagaatcgtcactacgctcacgttgattgtcctggtcacgctgattacgttaagaatatgattaccggagctgcacagatggacggagctatcctcgttgtttccggcgccgatggtcctatgcctcagactaaagagcatatccttttggctaagcaggttggtgttcctgatatggttgtgtttcttaacaaagaggatcaagtagatgatgcagagttgctagagctcgttgagcttgaggttcgtgagcttctctcgtcttatgaatttaacggtgatgatattccgattatctctggttctgctcttttagccgttgagactcttactgagaatcctaaggttaagagaggtgataacaaatgggtagataagatttatgaacttatggatgctgttgatgattacatccctatccctcagagacaaactgaattgccattcttgttagctgttgaggatgtgttctctatcactggacgtggtacggtggctacagggcgtgtcgagagaggtacggttaaggtaggagagactgtagatttagtgggtttgagggagactaggagttacactgtcactggggttgaaatgtttcagaagattcttgatgaggctttagctggtgacaatgtagggttgttgcttaggggtattcaaaaggctgatattcagagaggtatggttttagctaagccgggatcgattactccacataccaagtttgaagcaattatctatgtgttgaagaaagaggaaggtggaaggcattctccattctttgcagggtacaggcctcagttctacatgaggacgactgatgttacgggtaaagtgacgaagatcatgaacgacaaagatgaagagtcgaagatggttatgcccggtgatcgagtgaagattgttgttgagcttattgtgccggtggcttgtgaacaagggatgaggtttgctatcagagaaggaggaaagactgttggtgctggagttattgggacgatcctcgaatgattataaggttttagagagtttcatatagtgagcatctctacttgctttctttttgtttcactcttgattctcaaactctttgagtttttacctttttttatttatttttgtgccattttccataagaacttgaaatcaaaatgtcatatatttcttgattttctgatccttgttattcatctttgtctcgctccatatgattctgatgtggatttggtactcaacgaccaatcacttaatttg

SEQ ID NO: 6 - protein, 476 aa, Arabidopsis thaliana
MAISAPAACSSSSRILCSYSSPSPSLCPAISTSGKLKTLTLSSSFLPSYSLTTTSASQSTRRSFTVRAARGKFERKKPHVNIGTIGHVDHGKTTLTAALTMALASIGSSVAKKYDEIDAAPEERARGITINTATVEYETENRHYAHVDCPGHADYVKNMITGAAQMDGAILVVSGADGPMPQTKEHILLAKQVGVPDMVVFLNKEDQVDDAELLELVELEVRELLSSYEFNGDDIPIISGSALLAVETLTENPKVKRGDNKWVDKIYELMDAVDDYIPIPQRQTELPFLLAVEDVFSITGRGTVATGRVERGTVKVGETVDLVGLRETRSYTVTGVEMFQKILDEALAGDNVGLLLRGIQKADIQRGMVLAKPGSITPHTKFEAIIYVLKKEEGGRHSPFFAGYRPQFYMRTTDVTGKVTKIMNDKDEESKMVMPGDRVKIVVELIVPVACEQGMRFAIREGGKTVGAGVIGTILE

SEQ ID NO: 7 - DNA, 3675 bp, Nicotiana sylvestris
tgatcattctgcggattttagttatgcaagtgggcctaattcatcagaggaaagaaataacatgatcagcaacaactacccagctgatgtcagacaaattgtcaagaaactcgaggagctgcttggtcagcagcaaaaggagctaaatgatctcaggaaaaaacatgactcggctatatcagatattctaagcaagcttcctcctgagatccgtggcatttatggtttaaaaatatcttccaataatttgtagtggggatgaggctgctattctatggatcctgaagactgaagtttctctgtgaagatgtaagttactgtgcggccttttgccactcaactgcagatagtcttggattgcaccaattatctgactacccttgaaaggtttcaagatattcgagaatagctgaaagattaattttgccgacaatggtcacatgcagatcaaaagctagaagggaaaagcatggacctatcattagatacagattcacttctgtcaaggataccatcatttcagggccaggaattacagtaatacaggtttctcattacacgcatattgtgcgtctgcaagtcatgttgctgctaaaaatgagtcatggcatgtatacttgtacatatttacatgagtatttaactattggaaataccaattggttgaaataatgccagtcaatatataacttcttaagtttctttaattttatcttggtgttcactagatccgtaaaaaaggaaaacaatatatctgaatgattctactatttgctgttgtccggtttttcttcacaaagtatgttttttcatatgtgcttataaacgcccccgttttttttaaaactctgcttttacctatcctaaagctctttataagctccatacagatcattggtgtaaactcacggtttaaacttttgtgtataccctattgtcctggcactagtcaatcctttttactttagtatacatactagtatttgacatgaacatttttctatagcacacgttaccagatttttcttggcactctattgtatacatcccactccatgttaatttttaccaatctttatagcaaatatacagccattgttgcagatttggcatgtacaaccatgaggggcggagctagaatattccgagcgggttcggccaaatccagaacgactagaattccgaacccataagcttgaaatccttgctatgcctgtgataatcattaaattaaacactaccgggagaaaaagaaggctttggaacaaaaagatttttatttatttatgtgaagttaaaagaaaaggcagggtaaaaatttagactattttcttacgtgtcacattttgggaacagaattcacagaaatcccatacccctaatccccacagttgcaagttgcagcccagtgtgtcatcctagggaggtaagtattagataaaaccccacatatatgtgaggccctctttattcctaccaaggataacatcattatccttctctcctcttcttccttctttcaaccacaattctcactcccctctttcgtctctcttctccaacttcaatcccattttcaggcaaaaagctgtcatggcttcaatttcagcagcttctgccacagctacagcttctacaaagcttgcatacccttattccccttcttcctcaagcagcagcagcaacactgctgctgtattcccttcaaattcctcaaagcttatcctttcctcttcttttacacccaccccttcaacccttttcctccactcaccaacaactactccttccaccacccacccccgtcggttcactgtccgcgctgcacgtggcaaattcgagcgtaaaaaacctcacgtcaacattggtacaattggccacgttgaccatggaaagaccacactcacagctgctttgaccatggcgcttgcctctatgggcaactccgcccccaagaaatatgacgaaattgatgctgcccctgaagaaagggcgcgtggtattactatcaacactgccactgtggaatatgaaacggaaaacagacattatgcacacgtggactgcccggggcatgctgattatgtcaagaacatgattactggtgctgcccaaatggatggggcaattcttgttgtgtcaggtgctgatggcccaatgccacagactaaagagcatattttgttagctaagcaagttggggtccctaatatggttgttttcttgaacaaacaagaccaagttgatgatgaggagttacttgagcttgttgagttggaggtaagagaattattgtcaagttatgagttccctggtgatgaaattcctattatttctggttctgcacttttagctttagaggctttgatggctaatcctagtattaaaaggggtgaaaatcaatgggttgataagatttatcaattgatggataatgttgatgaatatatccctatcccacaaagacaaactgaattgcctttcttgatggctattgaggatgttttctcgattaccggtagaggtactgtggcgacggggagagtagagagagggactgttaaggttggggaaattgttgatatagttggattgaaggatactaggaatactacagtgacaggggttgagatgtttcagaagattttggatgaagcgatggcgggagataatgtgggattgttgttgagaggtattcagaagattgatattcagagagggatggtgttggcgaagcccggaacaattactccgcacacaaagtttgaagctttggtgtatgtgttgaagaaggaagagggaggaaggcattccccgttctttgcgggttataggcctcaattttacatgaggacaactgatgtgactggaaaggttactgtgattatgagtgacaaaggagaggaatctaagatggtcatgcctggcgatcgtgtaaacatggtggttgagcttatcatgccggttgcatgtgagcaagggatgaggtttgctatcagggaaggaggaaagactgttggagctggtgttattcagaaaatcttagaatgatgaacttgcagctgagcatctcttttcacatgatcggcactttccattgaagttacttaatccattgtcatatatgcaacttcttggttacttttattatgtcttagaatcttactttagtagaagtatcctgttttaaacaccaaattctactgaacttttgggatttttcggcagtctcctctttcatttttcctttgcttgaaaggaatagaacatttgatttcatgcactttatttaatttagaacaaatgtgcgactctgtttgaaaatttaagtgcagatttgctaatttgagattcactgcttcggctttttgtttcagcaaaatgatgctgatagaaaagcacaaaagaataaataatttgtttgtaaactatggataataatttgtcagctggccaatgactgaattttttatgtccatgtaactaaaaactatggtactcgaagcagatatgtatttatctgtattgtttaaaatcgacaagaggggttgctctgatggtaagcaacccccacttccaactaagaggctgtgagttcaagtttccccaagagcaaggtgggaagttcttggagggaaggatgccgagggtctaattggaaacagcctttctacc

SEQ ID NO: 8 - protein, 485 aa, Nicotiana sylvestris; identical to SEQ ID NO: 2

SEQ ID NO: 9 - DNA, 1678 bp, Oryza sativa
caaatccgaacctcccccccccccccactcctctgcgcctccctcctccgcggcctccgccatggcctccctcgcctccgcctccgcatccacctccctggtcttctccacctcctcctccaagccgcgcctcggctcctccgtcggattctcctcgcccgcgcggttccggcgcacggcggcggcggcggcgtccaagggcacggggcggcgcgcggggctgctggtaatgcgcgcggcgagggggaagttcgagcggaccaagccgcacgtcaacatcggcaccatcggccacgtcgaccacgggaagactacgctgacggcggcgctcaccatggtgctcgcctccgtgggcgggagcgcccccaagaagtacgacgagatcgacgcggcgcccgaggagcgcgcccgcggcatcaccatcaacaccgccaccgtcgagtacgagaccgagacccgccactacgcccacgtcgactgccccggccacgccgactacgtcaagaacatgattaccggcgccgcgcagatggacggcgccatcctcgtcgtctccggcgccgacgggcccatgccgcagaccaaggagcacatcctgctcgccaagcaggtcggtgtccccaagattgttgtcttcctcaacaagaaggaccaggtcgacgacgaggagctgctccagctcgtcgagctcgaggtccgcgaattgctctcctcctacgagtacgatggcgacgaagtgcccatcgtcgctggctccgcgctcaaggcgctcgagaacctcatggccaaccctgccattaagcgcggcgatgatgagtgggtggacgggatcttctcgttgattgattccgtggataactacatccctgtcccacagcgccagaccgacctcccgttcttgcttgctgttgaggatgtgttctccatcaccggtcgtggtaccgttgccactggccgtattgagcgtggcaccgtcaaggttggggacacggtcgatatcgtcggtatccgggagactcgcaactgcacggtgactggtgttgagatgttccagaagaccatggatgatgcgatggctggggacaatgtcggcctgcttctccgaggtatgcagaaggatgatatcgagagaggcatggtgcttgcgaagcctgcttccatcacgccacacaccaagtttgatgcggttgtgtatgtcctgaagaaagacgaaggtggacggcactcaccgtttttccctggttaccgccctcagttctacatgcggactaccgatgtgacggggaatgtcccaaagattatgaacgacaaggacgaggaggcgaagatgtgcatgcctggtgaccgtgtcaagatggttgtggagctcatccagcccgtcgcttgtgagcagggaatgaggtttgccatccctgagggtggaaagaccgtcggtgccggcgtcatcaatacgattttgaagtaaagtggatggagcatatccaccgtgagaattttccttgtttactttttgcgaaatgctctgtagttgttattacgcgttgagttttaggggttgatcatgtgaaattgtagtatggcacttttgttttcaagtgaatttgcatactttgtgatattcacgacaaagatacatgtgtttgcaactaatttggctatgcagtgccatttactgtcc

SEQ ID NO: 10 - protein, 467 aa, Oryza sativa
MASLASASASTSLVFSTSSSKPRLGSSVGFSSPARFRRTAAAAASKGTGRRAGLLVMRAARGKFERTKPHVNIGTIGHVDHGKTTLTAALTMVLASVGGSAPKKYDEIDAAPEERARGITINTATVEYETETRHYAHVDCPGHADYVKNMITGAAQMDGAILVVSGADGPMPQTKEHILLAKQVGVPKIVVFLNKKDQVDDEELLQLVELEVRELLSSYEYDGDEVPIVAGSALKALENLMANPAIKRGDDEWVDGIFSLIDSVDNYIPVPQRQTDLPFLLAVEDVFSITGRGTVATGRIERGTVKVGDTVDIVGIRETRNCTVTGVEMFQKTMDDAMAGDNVGLLLRGMQKDDIERGMVLAKPASITPHTKFDAVVYVLKKDEGGRHSPFFPGYRPQFYMRTTDVTGNVPKIMNDKDEEAKMCMPGDRVKMVVELIQPVACEQGMRFAIPEGGKTVGAGVINTILK

SEQ ID NO: 11 - DNA, 1365 bp, Arabidopsis thaliana
atggcgtccgttgttcttcgaaaccctagctcgaagcgccttgttccattctcttcccagatctactctcgctgtggtgcttccgttacttcctcttactcgatctctcattctatcggtggagatgatctctcttcctctaccttcggaacctcctccttctggagatccatggccacttttactcgaaataaacctcatgtaaatgttggaactattgggcatgttgatcatggcaagaccactttaactgctgcaatcacaaaggttcttgctgaggagggcaaagctaaagctattgcctttgatgaaattgataaagctcctgaagagaagaagagaggaattactattgccacggctcatgtggagtatgaaactgcaaagcgtcactatgctcatgtggattgccctggacacgcagattatgttaaaaatatgattactggagctgcgcaaatggatggcggaattcttgtggtttcaggaccagatggacccatgccgcagacaaaggaacatattctacttgcacgccaggttggtgttccctcacttgtgtgcttcttgaacaaagttgatgtggtggatgaccctgagctcttggagcttgtcgagatggaactacgtgagctcctcagcttctacaagtttcctggggatgatattcccatcatccgaggatctgctctgtccgcattacagggcaccaatgatgaaattggaaggcaagctatattaaagctgatggatgctgttgatgaatatatacctgaccctgttcgcgtccttgacaagcctttcttgatgccaattgaagatgttttctcaattcaaggacgtggaactgttgcaaccggtcgtatcgaacagggagtcattaaagtgggtgaagaagttgagatattgggtttacgtgaggggggtgttccactgaaatcgactgtaactggggttgagatgttcaagaagattttggataatggacaggctggtgataatgtaggacttcttctgcgtgggctaaagagagaagacattcagcgtggaatggtgattgctaagcctggttcatgcaagacatacaagaagtttgaagcagagatttacgtgctcacaaaggatgaaggtggacgtcacactgcatttttctctaactacaggcctcagttttacttgaggactgcagatatcactggcaaagtggaattacccgaaaacgtgaagatggttatgcctggtgacaatgtcacagctgttttcgagttaatcatgcctgtcccactcgaaacaggtcaaagatttgccttaagggaaggaggtagaacagttggagctggtgttgtatcaaaagtgatgacctaa

SEQ ID NO: 12 - protein, 454 aa, Arabidopsis thaliana
MASVVLRNPSSKRLVPFSSQIYSRCGASVTSSYSISHSIGGDDLSSSTFGTSSFWRSMATFTRNKPHVNVGTIGHVDHGKTTLTAAITKVLAEEGKAKAIAFDEIDKAPEEKKRGITIATAHVEYETAKRHYAHVDCPGHADYVKNMITGAAQMDGGILVVSGPDGPMPQTKEHILLARQVGVPSLVCFLNKVDVVDDPELLELVEMELRELLSFYKFPGDDIPIIRGSALSALQGTNDEIGRQAILKLMDAVDEYIPDPVRVLDKPFLMPIEDVFSIQGRGTVATGRIEQGVIKVGEEVEILGLREGGVPLKSTVTGVEMFKKILDNGQAGDNVGLLLRGLKREDIQRGMVIAKPGSCKTYKKFEAEIYVLTKDEGGRHTAFFSNYRPQFYLRTADITGKVELPENVKMVMPGDNVTAVFELIMPVPLETGQRFALREGGRTVGAGVVSKVMT

SEQ ID NO: 13 - DNA, 1425 bp, Arabidopsis thaliana
tagtttgagatggaagaaccagtagaccgagaagatatcgatttaagggtttcatccacagatataggctggaacgaattcgcggccgcctcgaagcgccttgttccattctcttcccagatctactctcgctgtggtgcttccgttacttcctcttactcgatctctcattctatcggtggagatgatctctcttcctctaccttcggaacctcctccttctggagatccatggccacttttactcgaaataaacctcatgtaaatgttggaactattgggcatgttgatcatggcaagaccactttaactgctgcaatcacaaaggttcttgctgaggagggcaaagctaaagctattgcctttgatgaaattgataaagctcctgaagagaagaagagaggaattactattgccacggctcatgtggagtatgaaactgcaaagcgtcactatgctcatgtggattgccctggacacgcagattatgttaaaaatatgattactggagctgcgcaaatggatggcggaattcttgtggtttcaggaccagatggacccatgccgcagacaaaggaacatattctacttgcacgccaggttggtgttccctcacttgtgtgcttcttgaacaaagttgatgtggtggatgaccctgagctcttggagcttgtcgagatggaactacgtgagctcctcagcttctacaagtttcctggggatgatattcccatcatccgaggatctgctctgtccgcattacagggcaccaatgatgaaattggaaggcaagctatattaaagctgatggatgctgttgatgaatatatacctgaccctgttcgcgtccttgacaagcctttcttgatgccaattgaagatgttttctcaattcaaggacgtggaactgttgcaaccggtcgtatcgaacagggagtcattaaagtgggtgaagaagttgagatattgggtttacgtgaaggggggtgttccactaaatcgactgtaactggggttgagatgttcaagaagattttggataatggacaggctggtgataatgtaggacttcttctgcgtgggctaaagagagaagacattcagcgtggaatggtgattgctaagcctggttcatgcaagacatacaagaagtttgaagcagagatttacgtgctcacaaaggatgaaggtggacgtcacactgcatttttctctaactacaggcctcagttttacttgaggactgcagatatcactggcaaagtggaattacccgaaaacgtgaagatggttatgcctggtgacaatgtcacagctgttttcgagttaatcatgcctgtcccactcgaaacaggtcaaagatttgccttaagggaaggaggtagaacagttggagctggtgttgtatcaaaagtgatgacctaa

SEQ ID NO: 14 - protein, 471 aa, Arabidopsis thaliana
MEEPVDREDIDLRVSSTDIGWNEFAAASKRLVPFSSQIYSRCGASVTSSYSISHSIGGDDLSSSTFGTSSFWRSMATFTRNKPHVNVGTIGHVDHGKTTLTAAITKVLAEEGKAKAIAFDEIDKAPEEKKRGITIATAHVEYETAKRHYAHVDCPGHADYVKNMITGAAQMDGGILVVSGPDGPMPQTKEHILLARQVGVPSLVCFLNKVDVVDDPELLELVEMELRELLSFYKFPGDDIPIIRGSALSALQGTNDEIGRQAILKLMDAVDEYIPDPVRVLDKPFLMPIEDVFSIQGRGTVATGRIEQGVIKVGEEVEILGLREGGCSTKSTVTGVEMFKKILDNGQAGDNVGLLLRGLKREDIQRGMVIAKPGSCKTYKKFEAEIYVLTKDEGGRHTAFFSNYRPQFYLRTADITGKVELPENVKMVMPGDNVTAVFELIMPVPLETGQRFALREGGRTVGAGVVSKVMT

SEQ ID NO: 15 - DNA, 1725 bp, Oryza sativa
cccgtctctctcctcctctcacctagcgccgccgccgccgccgccgcgaacgcgaacgcgatggccgcagccgcagtgctccggagccatggcgcccgccgcatcctctcctaccccaccctccgcgccgccgtgatctcgggccccacggcgctaccggatgcgtcggcggctgcggcggcggcgccccagcagccaccgcctctggctggaaccctgtgggcgaggtccatggccaccttcacccgcacgaagcctcatgtgaatgttggcaccattgggcacgtcgatcatggcaaaaccacactcactgctgcgattaccaaggtgttagctgaggcagggaaggctaaagctgttgcttttgacgagatcgacaaggccccggaggagaaagcaagaggaatcaccattgcaacggctcatgtggagtacgagactgctaaaaggcattatgctcatgtggactgcccagggcatgctgattatgtcaagaatatgatcactggagctgctcaaatggatggtggtattcttgtcgtatcagctcctgatggccccatgccacaaacgaaagagcatattcttctggcccgacaggttggtgtgccatcacttgtatgtttcttaaacaaagttgatgctgttgatgaccccgagctactagagcttgtagagatggagcttcgtgagctcctcagtttctacaagttccctggtgatgagattccaatcatccgtggatccgccttgtcagccttacaggggactaatgatgagattggaaagaatgccattttgaaactaatggatgcagttgacgagtatatccctgatcctgtgaggcagcttgataagtctttcttgatgccgatagaagatgttttctctatccagggccgtggaactgttgtgacagggcgtgttgagcaggggacaatcaaaactggtgaagatgttgagatcctaggtttgactccaagtggtcccttgaagactacagttactggtgttgagatgttcaaaaagatactggatcatggagaggctggtgacaatgttggtcttcttcttcgtggtcttaagcgtggtgatgtacagcgtggccaggtggtgtgcaaacctggtaccgttaagacgtaccaaaagtttgaggcggaaatttacgtccttacaaaggatgaaggtggtcgccacacagccttcttgtcaaattacagtccacagttctactttaggactgccgatgtcactggaaaagttgtgttacctgatggcgtggaaatggttatgcctggtgataatgtaactgcaggatttgagttgatatcacctgttccccttgagccaggccagagatttgcgctgagggaaggagggaggacagttggtgccggagttgtttccaaagtttacagctgatctgtttccagttttttctgcgtagcgaatttagtaacaaatgaatgttcatagggaatcagatctatccgaccggcaatatgagagcaaaagagcgagggactcgcagagcatcttctctgaattcatgagatgcagggatcgttggctcggccaccattccgaactagttattgcttttctttttccatgtctggttgttgctgtataacaacgcgtttagtcatcttaacacgtttttttgcccatagcttggtgactaaataagaaagattcatcataagcatcttaatggttgatgtt

SEQ ID NO: 16 - protein, 453 aa, Oryza sativa
MAAAAVLRSHGARRILSYPTLRAAVISGPTALPDASAAAAAAPQQPPPLAGTLWARSMATFTRTKPHVNVGTIGHVDHGKTTLTAAITKVLAEAGKAKAVAFDEIDKAPEEKARGITIATAHVEYETAKRHYAHVDCPGHADYVKNMITGAAQMDGGILVVSAPDGPMPQTKEHILLARQVGVPSLVCFLNKVDAVDDPELLELVEMELRELLSFYKFPGDEIPIIRGSALSALQGTNDEIGKNAILKLMDAVDEYIPDPVRQLDKSFLMPIEDVFSIQGRGTVVTGRVEQGTIKTGEDVEILGLTPSGPLKTTVTGVEMFKKILDHGEAGDNVGLLLRGLKRGDVQRGQVVCKPGTVKTYQKFEAEIYVLTKDEGGRHTAFLSNYSPQFYFRTADVTGKVVLPDGVEMVMPGDNVTAGFELISPVPLEPGQRFALREGGRTVGAGVVSKVYS

SEQ ID NO: 17 - DNA, 5993 bp, Zea mays
ggatcctacgaccttgtccagcgttgcccgcatctccgggctcatagccgccaggcaccgcaccgaccggctgcgctccgggggtgagagccacgcgccacgcctgagagcaaggcgctgggccggagcaggaggaatcagggagagcaaggcgaccctggcaggccgcgtccgcgtcgagagcgggagcgggacggcggtcccggcgagaggcctcgcggccgtggcggaggcagccatcgcggtgcgccggcggcgacggatggtacggaggcgcgtttgtggtgggctgagggctgtggacgggaaaaacggctgtgggctcctctagtagagctaataaattagcgtttggttgagctagctaataaactaatagttagttataagatagctaacaattaacagtgttattaacggagactgtttcaaactaaacaactaagttttagtagctaactattaattttagaggttccaaatagagcctaaaaccgttcgatggaaaacactacatatatattttcacaatttcatttaaaagttgctaaaacagatttagaggtgctttcagttttaccatacgagaaaatcggcttttaaaaaagctagtttctaaatccacttttttttgttaagcttttgatttttagggagcaaaatccaaaaccaaaagccataccaaatatacagttaggacggcatggaataattaattaaaccagtaataattatttttcgtggccgtggaggagaacggaggaaggttgtcgaagggagtcgagccgtcgaggcagcaagccgagtcgataccggagcggcccatgggccatgtatggtgttgtcttgggccgcaaatctgtgccttccagtccagcgtgccgcacggtatgcgtctgccggccgttctcattcccctccaaacttaaagccctaaacccatcttgctcccccctcgtctccactgctctgctgctcctcgcgccgccgcagcgacgtaatggcttccgccgcggtgctccggaacgccggctcccggcgcctcttctcctaccctactctccgcgccgctgcgatatatgtaccttctgcgctacccgatgcgcccgcggcggcggcagcgccggcccagccgccaccgacggccgggaccctctgggcgaggtctatggccaccttcacgcgcacgtgagtactccttggaccagatccgttccgtgcccctattttctcgttctagaatcactcatgagttcacttccattgatccgtgcaattcaatatccgtctattgcttcgtgtgattcactttgcaagtcgtgtcaatctgtggcaggaagccccatgtgaccgtcggcaccattgggcacgtcgatcacggcaaaaccactctcactgctgccattaccaaggttggatgtgttatactgttcctgcttcacatctcgcttcatttccaaacggatgggctgattctatggcgtctatttcctcgttattataggtcctggccgaggcagggaaggccaaagccgttgctttcgacgagatcgacaaggctccggaggagaaagccagaggaatcaccattgcgacagttgggtcctagcacgtctcctatagttgtgccagaccgatagagcaattctctttatattttttattcttcatttactgcccagtgtttaaccatttggattgtttttaggctcacgtcgagtatgagacggctaaaaggcattatgctcacgttgattgtccaggtcacgcagattatgtcaaggtttatacgaatcaagatttttttctttctcaaatgcattattattattttagtaatgaaggttgtttgcacgtcaaattgtgaaaaatctattgaaagccaccatctagagccattacattcctttgtttatggtccaagtctttttgtttttgtgaaaatggtagaacatgatcactggagctgctcaaatggatggtggtattcttgtcgtgtcagctcctgatggcccgatgccacaaactaaggagcatattcttcttgcccgtcaggtatggaatgcttgtttctattgttgtttaaaatgcttctttcaggctctgcttgtatagtctcgacttgatcagataaacatgagctttctttttaggttggtgtaccttcccttgtgtgcttcttaaacaaagttgatgctgttgatgatccagagcttctggaacttgtagagatggagcttcggggtatgctaccattcatgtctagacatcatgtttagattcctgtatgtcttctgtattttttaaggttgtcttcttggtccccctccccctcttttttctgcagagctcctcagtttctacaagttccccggtgatgagattccgatcatccgtgggtctgcgttgtctgctttgcaagggaacaatgatgagataggaaagaatgcaattttgaaattaatggacgcagttgatgagtacatccctgatcctgtgaggcagcttgataagcctttcttgatgccgatagaagatgtgttttctattcaggtgaatttatttatgagctatcactactgcagtatgcattagctattcaattatgatgtttgatgtttcttcgacgttgattagggatactagttttgtttttcttgggctactgggtccatcaaaacttaccaagtcagaatacttttgaattatggctatagggatgcaatgcaaatatgcaatgaattaaagtggtgacccaactaaatgctgagattttcagtaccgctgtattaagcattgtatattccctatttgtgtgaataaatgtttctattcagcatcttcttttgttaaactaattaagattgcaccaagaaacagtttaaagtttgcatagattgaccgcttgcatttgctgttgaatgaaaacacataaaagtattagtttgtgttgtttgaaataaggttgttaaagagatcactggtaaatgtgttgccatggagattgtgtcaatatatgaaaatgcatatattttatcggattaatagaaaatgaaaaggaaaaacattgataggactgtactgtcaatcaggtcatatgctttttttatatttagtaattatttgtgtggcggccattcttgtttccagttacatactgtacaacgaggattcttcttgttcttatattcctactgtctaagaaccaaagggttgtttctagtgacgccgttgggcaaaaatttggttacccagtccccatacctgaaaatcatccaccccgctttatccatccccagaactagttgataatgcccaatgggcatggatgacccaatgcgtaagcagtgacggctatcattaccctcaccgcaaccaccggctgcaccatggtcttgttggacactctgctgtgcacctccttgttgcaaattagcctgtgcgcagcgcagggactaaacacagatgctgatgtgggtgtccacagccaccttcaaagcctgctgctgttgccaccgttgtcagctgtcacacaagcgctcggcggcggccagggacttagacagtggcggccccattaaacaggggtggcactggcaggcaagtggacagtggacatctgcaactccatcaaaggtatcaatgtctggtgttggcgtgacttttggcagaagcacctgtgccggcattgaggacttgaaacgccttgccgcccacttgctgctggggccgctgctgtcgttgtcggtggcgactggggccttggtttgacagatgtagatggcccatggtgcttggtgctggtggctgcagggagggccgtaagcagaatagaccaggtgattggggaagcgggtttcccatgttttatctacccatacccaattttagatggataatccataccatacccaaacagaatgggtatggatttggttgaagggtaacaaatggcaacattagtgatttcagtagacattctttattataccatatttgtggttttgtgctctttctgacatgttttgttttcttttagtattttaaagttttttcgttttcatttacaatttcctcccttgtgctgtcatgttcttaaagtaaacatgaattgaattgctgatggagctattaagcattgcaatgcaatgcaatggccatttaatcaaatgtttttttgtgagggagttattataagtttttaagtagcctgtctgatctgcagggtcgtggaactgttgttactgggcgtgttgaacagggaacaatcaaaactggtgaagatgttgagatcttgggtttggcacaggttagatcctttttgaactgaattactgcatgtttatcctagtttgtagtgtttgaagtacggattgaatgagtttgtggtttataaaatttagtatgttagtactccttaattataaggtgtttttggcttttcaagatacattgtttttattgtgtatctagacatagtgcatatctaagtgcatagcaaaaattgtatttaaacatggagctctaacttagtatacaacctgccatttccttgactgtctaacttttggatgatttatttgtaaatgtaaggcaataactaatcacaagagcaggaggttctaatatttctttttctggtttagtacccactgcacctatatatgttgtgacgaaatatattattggttgcagactggtcccttgaagactacagttactggtgttgagatgttcaaaaagatactggaccatgggaaagtaatgattctgaaagcatagtgtgtgaggctttctattgatccaaattatcatacatgaaacttcttgtgtacaggctggtgacaacgttgggcttcttctccgtggtcttaagcgtggtgatgttgagcgtggccaggttagatttcctgaattcgaaattgtctgagtaagtttttcgaaatattcattgaactgttttgcctggaaaacgccttgcatttgatggtacatggttgcgtgatcaccaaacaaattatacagttctcttaatcctgatcctctgtatgttcctcaatttctgggtgtccaatatcgaaactgaacgatctcgaatgttgacaccctgtcgtttcctaccgtttcaggtggtgtgcagacctgggtattctgaagactgcaaaaagttcgaggcggaaatatatgtcctgacaaaggatgaaggtggccggcacactgcctttgtcacaaattacagcccccaattctacttcaggacagctgatgtcactggaagggttgagctgcttggggagatgaagatggttttgcccggtgacaatgtaactgcgaattttgacttgatatcacctgttcctcttgaacctggtaacgattacattctcaatttctttcgacggtatagaactatagaccaggtcttctgacaacatgctgttcttcgtgatgccattgcacgctccaagctacttcttttcattgaatactaaagaaattcatattggatcgtcaactccgcctttgatggcagggcaaagatttgcgattagggaaggaggaagaacggtcggtgctggagtcgtctccaaagttcttagctgatcagtttccactttcttctccgtagcaattcagtgaacaaacgagtgttgatatgtccgaccgggttagaaacgagcgcaaagcaccgggttttcttttgcatttttcttggaatgcctgagcaaaggcagagctggcctggccagtggccaccattttattgttgaggggcgtggtgctgtgtacaccacggttagtttggttgacacgcaagttgggcataactttgattgaataatactagtctcatcataaacatattttctagcaattttgtcatgtcggtcttggaggcgctttgtcaagtccgcgaaaaaaaatgctggttggactatatatgtgtatgtatttgtgatgtttaccgtcgctagcttctgatctggtgatgc

SEQ ID NO: 18 - protein, 452 aa, Zea mays
MASAAVLRNAGSRRLFSYPTLRAAAIYVPSALPDAPAAAAAPAQPPPTAGTLWARSMATFTRTKPHVTVGTIGHVDHGKTTLTAAITKVLAEAGKAKAVAFDEIDKAPEEKARGITIATAHVEYETAKRHYAHVDCPGHADYVKNMITGAAQMDGGILVVSAPDGPMPQTKEHILLARQVGVPSLVCFLNKVDAVDDPELLELVEMELRELLSFYKFPGDEIPIIRGSALSALQGNNDEIGKNAILKLMDAVDEYIPDPVRQLDKPFLMPIEDVFSIQGRGTVVTGRVEQGTIKTGEDVEILGLAQTGPLKTTVTGVEMFKKILDHGKAGDNVGLLLRGLKRGDVERGQVVCRPGYSEDCKKFEAEIYVLTKDEGGRHTAFVTNYSPQFYFRTADVTGRVELLGEMKMVLPGDNVTANFDLISPVPLEPGQRFAIREGGRTVGAGVVSKVLS

SEQ ID NO: 19 - DNA, 1350 bp, Arabidopsis thaliana
atgggtaaagagaagtttcacatcaacattgtggtcattggccacgtcgattctggaaagtcgaccaccactgggcacttgatctacaagttgggtggtattgacaagcgtgtcattgagaggttcgagaaggaggctgctgagatgaacaagaggtccttcaagtacgcatgggttttggacaaacttaaggctgagcgtgagcgtggtatcaccattgacattgctctctggaagttcgagaccaccaagtactactgcactgtcattgatgctcctggccatcgtgatttcatcaagaacatgatcactggtacctcccaggctgattgtgctgtccttatcattgactccaccactggtggttttgaggctggtatctccaaggatggtcagacccgtgagcacgctctacttgctttcacccttggtgtcaagcagatgatctgctgttgtaacaagatggatgccactacccccaagtactccaaggccaggtacgatgaaatcatcaaggaggtgtcttcctacttgaagaaggttggttacaaccccgacaaaatcccatttgtgcccatctctggatttgagggtgacaacatgattgagaggtccaccaaccttgactggtacaagggaccaactctccttgaggctcttgaccagatcaacgagcccaagaggccgtcagacaagccccttcgtctcccacttcaggatgtctacaagattggtggtattggaacggtgccagtgggacgtgttgagactggtatgatcaagcctggtatggttgtgacctttgctcccacaggattgaccactgaggtcaagtctgttgagatgcaccacgagtctcttcttgaggcacttccaggtgacaacgttgggttcaatgttaagaatgttgctgtcaaggatcttaagagagggtacgtcgcatccaactccaaggatgaccctgccaagggtgctgctaacttcacctcccaggtcatcatcatgaaccaccctggtcagattggtaacggttacgccccagtcctggattgccacacctctcacattgcagtcaagttctctgagatcttgaccaagattgacaggcgttctggtaaggagattgagaaggagcctaaattcttgaagaatggtgatgctggtatggtgaagatgactccaaccaagcccatggttgtggagaccttctctgagtacccaccacttggacgtttcgctgtgagggacatgaggcagactgttgcagtcggtgttatcaagagtgttgacaagaaggacccaactggagccaaggttaccaaggctgcagtcaagaagggtgccaagtga

SEQ ID NO: 20 - protein, 449 aa, Arabidopsis thaliana
MGKEKFHINIVVIGHVDSGKSTTTGHLIYKLGGIDKRVIERFEKEAAEMNKRSFKYAWVLDKLKAERERGITIDIALWKFETTKYYCTVIDAPGHRDFIKNMITGTSQADCAVLIIDSTTGGFEAGISKDGQTREHALLAFTLGVKQMICCCNKMDATTPKYSKARYDEIIKEVSSYLKKVGYNPDKIPFVPISGFEGDNMIERSTNLDWYKGPTLLEALDQINEPKRPSDKPLRLPLQDVYKIGGIGTVPVGRVETGMIKPGMVVTFAPTGLTTEVKSVEMHHESLLEALPGDNVGFNVKNVAVKDLKRGYVASNSKDDPAKGAANFTSQVIIMNHPGQIGNGYAPVLDCHTSHIAVKFSEILTKIDRRSGKEIEKEPKFLKNGDAGMVKMTPTKPMVVETFSEYPPLGRFAVRDMRQTVAVGVIKSVDKKDPTGAKVTKAAVKKGAK

SEQ ID NO: 21 - DNA, 1200 bp, Synechocystis
atggcacgcgcaaaatttgaacggacgaaagaccacgtaaacattggcaccattggtcacgttgaccacggtaaaaccaccctaacggcggcgatcaccatgaccttggcggaattgggtggtgccaaagcccggaaatatgaagatattgacgcggctcctgaggaaaaagcccggggcattaccatcaatactgcccacgttgagtatgaaaccgatagccgtcactatgcccacgtagactgtcctggtcacgctgactatgtgaaaaacatgatcaccggtgctgcccagatggacggtgcgattctggtggtttccgccgctgatggccccatgccccaaacccgggaacacattcttttggctaagcaagtaggggttcccaaactggtggtctttttgaataagaaagacatggtggacgacgaagaactgctggaattggtggaactggaagttcgtgaattgctcagcgactacgatttccccggtgatgacattcccatcgttgctggctctgccctcaaggcgatcgaaggagaaaaagaatacaaagatgcgattcttgaactcatgaaagccgttgacgactacatcgacacccctgagcgggaagttgataagcccttcttgatggcggtggaagacgtattctccatcactggtcgtggtaccgttgccaccggtcggattgagcgcggaaaagttaaagttggcgaagaaatttccatcgtcggcattaaagatacccgtaaagccactgttaccggtgtggaaatgttccaaaaaacccttgaagagggaatggctggggacaacgtgggtctgctcctccgggggattcaaaaagaggacatcgaacggggtatggttttggctaaacccggttccatcactccccacaccgaatttgaaggggaagtttacgtgctcaagaaagaagaaggtggccgccacactcctttctttgccaactaccgccctcagttctatgtacggaccactgacgtaaccggtaccatcaaaagctacaccgctgacgacggcagtgctgtggaaatggttatgcctggggatcgtatcaaaatgaccgttgagctcattaacccgatcgccattgaacaggggatgcgttttgctatccgcgaaggtggtcgtaccatcggcgccggtgttgtttctaagattttgaagtag

SEQ ID NO: 22 - protein, 399 aa, Synechocystis
MARAKFERTKDHVNIGTIGHVDHGKTTLTAAITMTLAELGGAKARKYEDIDAAPEEKARGITINTAHVEYETDSRHYAHVDCPGHADYVKNMITGAAQMDGAILVVSAADGPMPQTREHILLAKQVGVPKLVVFLNKKDMVDDEELLELVELEVRELLSDYDFPGDDIPIVAGSALKAIEGEKEYKDAILELMKAVDDYIDTPEREVDKPFLMAVEDVFSITGRGTVATGRIERGKVKVGEEISIVGIKDTRKATVTGVEMFQKTLEEGMAGDNVGLLLRGIQKEDIERGMVLAKPGSITPHTEFEGEVYVLKKEEGGRHTPFFANYRPQFYVRTTDVTGTIKSYTADDGSAVEMVMPGDRIKMTVELINPIAIEQGMRFAIREGGRTIGAGVVSKILK

SEQ ID NO: 23 - protein, 32 aa, Chlamydomonas
MAMAMRSTFAARVGAKPAVRGARPASRMSCMA

SEQ ID NO: 24 - protein, 32 aa, Chlamydomonas
MQVTMKSSAVSGQRVGGARVATRSVRRAQLQV

SEQ ID NO: 25 - protein, 52 aa, Arabidopsis thaliana
MASLMLSLGSTSLLPREINKDKLKLGTSASNPFLKAKSFSRVTMTVAVKPSR

SEQ ID NO: 26 - protein, 54 aa, Arabidopsis thaliana
MATQFSASVSLQTSCLATTRISFQKPALISNHGKTNLSFNLRRSIPSRRLSVSC

SEQ ID NO: 27 - protein, 70 aa, Arabidopsis thaliana
MASIAASASISLQARPRQLAIAASQVKSFSNGRRSSLSFNLRQLPTRLTVSCAAKPETVDKVCAVVRKQL

SEQ ID NO: 28 - protein, 74 aa, Arabidopsis thaliana
MASIATSASTSLQARPRQLVIGAKQVKSFSYGSRSNLSFNLRQLPTRLTVYCAAKPETVDKVCAVVRKQLSLKE

SEQ ID NO: 29 - DNA, 1236 bp, Oryza sativa
ggtcagccaatacattgatccgttgccaatcatgcaaagtattttggctgtggccgagtgccggaattgataattgtgttctgactaaattaaatgaccagaagtcgctatcttccaatgtatccgaaacctggattaaacaatcctgttctgttctctagcccctcctgcatggccggattgtttttttgacatgttttcttgactgaggcctgtttgttctaaactttttcttcaaacttttaactttttcatcacatcagaacttttctacacatataaacttttaacttttccgtcacatcgttccaatttcaatcaaactttcaattttggcgtgaactaaacacaccctgagtcttttattgctcctccgtacgggttggctggttgagaataggtattttcagagagaaaatctagatattgggaggaacttggcatgaatggccactatatttagagcaattctacggtccttgaggaggtaccatgaggtaccaaaattttagtgtaaattttagtatctcattataactaggtattatgaggtaccaaatttacaatagaaaaaatagtacttcatggtactttcttaagtaccgtaaaattgctcctatatttaaggggatgtttatatctatccatatccataatttgattttgataagaaaaaatgtgagcacaccaagcatgtccatgaccttgcactcttggctcactcgtcaactgtgaagaacctcaaaaatgctcaatatagctacaggtgcctgaaaaaataactttaaagttttgaacatcgatttcactaaacaacaattattatctccctctgaaagatgatagtttagaactctagaatcattgtcggcggagaaagtaaattattttccccaaatttccagctatgaaaaaaccctcaccaaacaccatcaaacaagagttcaccaaaccgcccatgcggccatgctgtcacgcaacgcaccgcattgcctgatggccgctcgatgcatgcatgcttccccgtgcacatatccgacagacgcgccgtgtcagcgagctcctcgaccgacctgtgtagcccatgcaagcatccacccccgccacgtacaccccctcctcctccctacgtgtcaccgctctctccacctatatatgcccacctggcccctctcctcccatctccacttcacccgatcgcttcttcttcttcttcgttgcattcatcttgctagc

(19) United States
(12) Patent Application Publication
(10) Pub. No.: US 2008/0115239 A1
(43) Pub. Date: May 15, 2008

(54) Plants Having Increased Yield And Method For Making The Same

(75) Inventor: Christophe Reuzeau, La Chapelle Gonaguet (FR)

Correspondence Address: CONNOLLY BOVE LODGE & HUTZ, LLP, P O BOX 2207, WILMINGTON, DE, 19899, US

(73) Assignee: Crop Design N.N., Zwijnaarde (BE)

(21) Appl. No.: 11/793,684

(22) PCT Filed: Dec. 23, 2005
(86) PCT No.: PCT/EP05/57154

§ 371 Date: Jul. 5, 2007

Related U.S. Application Data
(60) Provisional application No. 60/641,657, filed on Jan. 6, 2005.

(30) Foreign Application Priority Data
Dec. 24, 2004 (EP) 04106984.0

(51) Int. Cl. C12N 15/82; A01H 1/06; A01H 5/00; C12Q 1/68; C12N 15/00

(52) U.S. Cl. 800/276; 435/320.1; 435/6; 800/278; 800/290; 800/320; 800/320.1; 800/320.2; 800/320.3

(57) ABSTRACT

The present invention concerns a method for increasing plant yield in plants grown under non-stress conditions, by preferentially increasing activity in a plastid of an EFTu polypeptide or a homologue thereof. One such method comprises introducing into a plant an EFTu nucleic acid or variant thereof. The invention also relates to transgenic plants having introduced therein an EFTu nucleic acid or variant thereof, which plants have increased yield, particularly increased seed yield, relative to corresponding wild type plants. The present invention also concerns constructs useful in the methods of the invention.

The present invention relates generally to the field of molecular biology and concerns a method for increasing yield in a plant relative to corresponding wild type plants. More specifically, the present invention concerns a method for increasing plant yield under non-stressed conditions by preferentially increasing activity in a plastid of a translation elongation factor (EFTu) or a homologue thereof. The present invention also concerns plants having preferentially increased activity in a plastid of an EFTu or a homologue thereof, which plants have increased yield under non-stressed conditions relative to corresponding wild type plants grown under comparable conditions. The invention also provides constructs useful in the methods of the invention.

The ever-increasing world population and the dwindling supply of arable land available for agriculture fuel research towards improving the efficiency of agriculture. Conventional means for crop and horticultural improvements utilise selective breeding techniques to identify plants having desirable characteristics. However, such selective breeding techniques have several drawbacks, namely that these techniques are typically labour intensive and result in plants that often contain heterogeneous genetic components that may not always result in the desirable trait being passed on from parent plants. Advances in molecular biology have allowed mankind to modify the germplasm of animals and plants. Genetic engineering of plants entails the isolation and manipulation of genetic material (typically in the form of DNA or RNA) and the subsequent introduction of that genetic material into a plant. Such technology has the capacity to deliver crops or plants having various improved economic, agronomic or horticultural traits. A trait of particular economic interest is yield. Yield is normally defined as the measurable produce of economic value from a crop. This may be defined in terms of quantity and/or quality. Yield is directly dependent on several factors, for example, the number and size of the organs, plant architecture (for example, the number of branches), seed production and more. Root development, nutrient uptake and stress tolerance may also be important factors in determining yield. Crop yield may therefore be increased by optimizing one of the abovementioned factors. Seed yield is also a trait of particular economic interest with plant seeds being an important source of human and animal nutrition. Crops such as, corn, rice, wheat, canola and soybean account for over half of total human caloric intake, whether through direct consumption of the seeds themselves or through consumption of meat products raised on processed seeds. They are also a source of sugars, oils and many kinds of metabolites used in industrial processes. Seeds contain an embryo, the source of new shoots and roots after germination, and an endosperm, the source of nutrients for embryo growth, during germination and early growth of seedlings. The development of a seed involves many genes, and requires the transfer of metabolites from roots, leaves and stems into the growing seed. The endosperm, in particular, assimilates the metabolic precursors of carbohydrate polymers, oil and proteins and synthesizes them into storage macromolecules to fill out the grain. The ability to increase plant seed yield, whether through seed number, seed biomass, seed development, seed filling, or any other seed-related trait would have many applications in agriculture, and even many non-agricultural uses such as in the biotechnological production of substances such as pharmaceuticals, antibodies or vaccines.

It has now been found that preferentially increasing activity in a plastid of a translation elongation factor (EFTu) gives plants grown under non-stressed conditions increased yield relative to corresponding wild type plants.

In plants, protein synthesis occurs in three sub cellular compartments, namely the cytoplasm, plastids and mitochondria. The mechanisms responsible for protein synthesis in the cytoplasm, plastids and mitochondria are distinct from each other. Plant cells therefore contain three different types of ribosomes, three groups of transfer RNAs (tRNAs), and three sets of auxiliary factors for protein synthesis. Plastids and mitochondria are thought to have arisen through the endosymbiosis of ancient prokaryotic organisms. Consistent with this theory, the protein synthetic machinery in plastids and mitochondria is more closely related to bacterial systems than to the translation apparatus in the surrounding plant cell cytoplasm.

Translation elongation factors (EFTus) are essential components of protein synthesis playing a role in polypeptide elongation. EFTu interacts with aminoacyl tRNA and transports the codon-specific tRNA to the aminoacyl site on the ribosome (ribosomal A-site) during the translation elongation step. EFTu is encoded in the chloroplast of lower photosynthetic eukaryotes such as Chlamydomonas and Euglena, whereas in higher plants an evolutionary transfer of these genes occurred from the chloroplast to the nucleus. Several cDNA clones encoding chloroplast and other EFTus have been identified in a number of higher plants.

US published patent application US 2003/0044972 A1 in the name of Ristic et al. describes a heat shock protein with high homology to chloroplast elongation factor EFTu and the temporal and spatial expression of the heat shock protein in a plant organ or tissue to enhance tolerance to heat and drought in female reproductive organs.

Bhadula et al. (Planta (2001)212: 359-366) show the heat-stress induced synthesis of EFTu in a heat-tolerant maize line.

While it is apparent from the prior art that EFTu plays a protective role during heat stress, it is not apparent from the prior art that EFTu would give any beneficial effects under non-stressed or normal growth conditions.

It has now surprisingly been found that preferentially increasing activity in a plastid of an EFTu or a homologue thereof gives plants grown under non-stressed conditions increased yield relative to wild type plants grown under corresponding conditions.

Reference herein to “wild type plants” is taken to mean any suitable control plant(s), the choice of which would be within the capabilities of a person skilled in the art and may include, for example, corresponding wild type plants or corresponding plants without the gene of interest. A “control plant” or “wild type plant” as used herein refers not only to whole plants, but also to plant parts, including seeds and seed parts.

Reference herein to “non-stressed conditions” is taken to mean growth/cultivation of a plant at any stage in its life cycle (from seed to mature plant and back to seed again) under normal growth conditions which include the everyday mild stresses that every plant encounters, but which does not include severe stress. Plants typically respond to exposure to stress by growing more slowly. In conditions of severe stress, the plant may even stop growing altogether. Mild stress on the other hand is defined herein as being any stress to which a plant is exposed which does not result in the plant ceasing to grow altogether without the capacity to resume growth. One example of a severe stress that is specifically excluded is temperatures above 35° C. as measured in the shade by a thermometer housed in an instrument shelter that is away from materials that may absorb heat and affect an accurate air temperature reading. Another example of a severe stress that is specifically excluded is drought stress, which is defined herein as a continuous increase in the degree of dryness over a period of seven days in comparison to a “normal” or average amount. Such normal or average amounts will vary from region to region.

According to the present invention, there is provided a method for increasing plant yield under non-stressed conditions relative to corresponding wild type plants grown under comparable conditions, comprising preferentially increasing activity in a plastid of an EFTu polypeptide or a homologue thereof.

Advantageously, performance of the methods according to the present invention result in plants having increased yield, especially increased seed yield.

The term “increased yield” as defined herein is taken to mean an increase in any one or more of the following obtained under non-stressed conditions, each relative to corresponding wild type plants: (i) increased biomass (weight) of one or more parts of a plant, particularly aboveground (harvestable) parts, increased root biomass or increased biomass of any other part; (ii) increased seed yield, which may result from an increase in seed biomass (seed weight) and which may be an increase in the seed weight per plant or on an individual seed basis, and which increase in seed weight may be due to altered seed dimensions, such as seed length and/or seed width and/or seed area and/or seed perimeter; (iii) increased number of flowers (florets) per panicle, which is expressed as a ratio of number of filled seeds over number of primary panicles; (iv) increased seed fill rate (which is the number of filled seeds divided by the total number of seeds and multiplied by 100); (v) increased number of (filled) seeds; (vi) increased seed size; (vii) increased seed volume; (viii) increased harvest index, which is expressed as a ratio of the yield of harvestable parts, such as seeds, over the total biomass; and (ix) increased thousand kernel weight (TKW), which is extrapolated from the number of filled seeds counted and their total weight; an increased TKW may result from an increased seed size and/or seed weight. An increased TKW may also result from an increase in embryo size and/or endosperm size.

Taking corn as an example, a yield increase may be manifested as one or more of the following: increase in the number of plants per hectare or acre, an increase in the number of ears per plant, an increase in the number of rows, number of kernels per row, kernel weight, thousand kernel weight, ear length/diameter, among others. Taking rice as an example, a yield increase may be manifested by an increase in one or more of the following: number of plants per hectare or acre, number of panicles per plant, number of spikelets per panicle, number of flowers per panicle, increase in the seed filling rate, increase in thousand kernel weight, among others. An increase in yield may also result in modified architecture, or may occur because of modified architecture.

According to a preferred feature, performance of the methods of the invention result in plants having increased seed yield, such as defined in (ii) to (ix) above. Therefore, according to the present invention, there is provided a method for increasing seed yield in a plant grown under non-stressed conditions, which method comprises preferentially increasing activity in a plastid of an EFTu polypeptide or a homologue thereof.

Since the transgenic plants according to the present invention have increased yield, it is likely that these plants exhibit an increased growth rate (during at least part of their life cycle), relative to the growth rate of corresponding wild type plants at a corresponding stage in their life cycle. The increased growth rate may be specific to one or more parts of a plant (including seeds), or may be throughout substantially the whole plant. A plant having an increased growth rate may even exhibit early flowering. The increase in growth rate may take place at one or more stages in the life cycle of a plant or during substantially the whole plant life cycle. Increased growth rate during the early stages in the life cycle of a plant may reflect enhanced vigour. The increase in growth rate may alter the harvest cycle of a plant allowing plants to be sown later and/or harvested sooner than would otherwise be possible. If the growth rate is sufficiently increased, it may allow for the sowing of further seeds of the same plant species (for example sowing and harvesting of rice plants followed by sowing and harvesting of further rice plants all within one conventional growing period). Similarly, if the growth rate is sufficiently increased, it may allow for the sowing of further seeds of different plants species (for example the sowing and harvesting of rice plants followed by, for example, the sowing and optional harvesting of soybean, potato or any other suitable plant). Harvesting additional times from the same rootstock in the case of some plants may also be possible. Altering the harvest cycle of a plant may lead to an increase in annual biomass production per acre (due to an increase in the number of times (say in a year) that any particular plant may be grown and harvested). An increase in growth rate may also allow for the cultivation of transgenic plants in a wider geographical area than their wild-type counterparts, since the territorial limitations for growing a crop are often determined by adverse environmental conditions either at the time of planting (early season) or at the time of harvesting (late season). Such adverse conditions may be avoided if the harvest cycle is shortened. The growth rate may be determined by deriving various parameters from growth curves, such parameters may be: T-Mid (the time taken for plants to reach 50% of their maximal size) and T-90 (time taken for plants to reach 90% of their maximal size), amongst others.

Performance of the methods of the invention gives plants having an increased growth rate. Therefore, according to the present invention, there is provided a method for increasing the growth rate of plants grown under non-stressed conditions, which method comprises preferentially increasing activity in a plastid of an EFTU-like polypeptide or a homologue thereof.

The abovementioned growth characteristics may advantageously be modified in any plant.

The term “plant” as used herein encompasses whole plants, ancestors and progeny of plants and plant parts (such as plant cells, seeds, shoots, stems, leaves, roots, flowers and tubers), tissues and organs, wherein each of the aforementioned comprise the gene/nucleic acid of interest. The term “plant” also encompasses suspension cultures, callus tissue, embryos, meristematic regions, gametophytes, sporophytes, pollen, and microspores, again wherein each of the aforementioned comprise the gene/nucleic acid of interest.

Plants that are particularly useful in the methods of the invention include all plants which belong to the superfamily Viridiplantae, in particular monocotyledonous and dicotyledonous plants including fodder or forage legumes, ornamental plants, food crops, trees or shrubs selected from the list comprising Acacia spp., Acer spp., Actinidia spp., Aesculus spp., Agathis australis, Albizia amara, Alsophila tricolor, Andropogon spp., Arachis spp, Areca catechu, Astelia fragrans, Astragalus cicer, Baikiaea plunjuga, Betula spp., Brassica spp., Bruguiera gymnorrhiza, Burkea africana, Butea frondosa, Cadaba farinosa, Calliandra spp, Camellia sinensis, Canna indica, Capsicum spp., Cassia spp., Centroema pubescens, Chaenomeles spp., Cinnamomum cassia, Coffea arabica, Colophospermum mopane, Coronillia varia, Cotoneaster serotina, Crataegus spp., Cucumis spp., Cupressus spp., Cyathea dealbata, Cydonia oblonga, Cryptomeria japonica, Cymbopogon spp., Cynthea dealbata, Cydonia oblonga, Dalbergia monetaria, Davallia divaricata, Desmodium spp., Dicksonia squarosa, Diheteropogon amplectens, Dioclea spp, Dolichos spp., Dorycnium rectum, Echinochloa pyramidalis, Ehrartia spp., Eleusine coracana, Eragrestis spp., Erythrina spp., Eucalyptus spp., Euclea schimperi, Eulalia villosa, Fagopyrum spp., Feijoa sellowiana, Fragaria spp., Flemingia spp, Freycinetia banksii, Geranium thunbergii, Ginkgo biloba, Glycine javanica, Gliricidia spp, Gossypium hirsutum, Grevillea spp., Guibourtia coleosperma, Hedysarum spp., Hemarthia altissima, Heteropogon contortus, Hordeum vulgare, Hyparrhenia rufa, Hypericum erectum, Hyperthelia dissoluta, Indigo incamata, Iris spp., Leptarrhena pyrolifolia, Lespediza spp., Lettuca spp., Leucaena leucocephala, Loudetia simplex, Lotonus bainesil, Lotus spp., Macrotyloma axillare, Malus spp., Manihot esculenta, Medicago sativa, Metasequoia glyptostroboides, Musa sapientum, Nicotianum spp., Onobrychis spp., Ornithopus spp., Oryza spp., Peltophorum africanum, Pennisetum spp., Persea gratissima, Petunia spp., Phaseolus spp., Phoenix canariensis, Phormium cookianum, Photinia spp., Picea glauca, Pinus spp., Pisum sativum, Podocarpus totara, Pogonarthria fleckii, Pogonarthria squaffosa, Populus spp., Prosopis cineraria, Pseudotsuga menziesil, Pterolobium stellatum, Pyrus communis, Quercus spp., Rhaphiolepsis umbellata, Rhopalostylis sapida, Rhus natalensis, Ribes grossularia, Ribes spp., Robinia pseudoacacia, Rosa spp., Rubus spp., Salix spp., Schyzachyrium sanguineum, Sciadopitys verticillata, Sequoia sempervirens, Sequoiadendron giganteum, Sorghum bicolor, Spinacia spp., Sporobolus fimbriatus, Stiburus alopecuroides, Stylosanthos humilis, Tadehagi spp, Taxodium distichum, Themeda triandra, Trifolium spp., Triticum spp., Tsuga heterophylla, Vaccinium spp., Vicia spp., Vitis vinifera, Watsonia pyramidata, Zantedeschia aethiopica, Zea mays, amaranth, artichoke, asparagus, broccoli, Brussels sprouts, cabbage, canola, carrot, cauliflower, celery, collard greens, flax, kale, lentil, oilseed rape, okra, onion, potato, rice, soybean, strawberry, sugar beet, sugarcane, sunflower, tomato, squash, tea and algae, amongst others. According to a preferred embodiment of the present invention, the plant is a crop plant such as soybean, sunflower, canola, alfalfa, rapeseed, cotton, tomato, potato or tobacco. Further preferably, the plant is a monocotyledonous plant, such as sugarcane. More preferably, the plant is a cereal, such as rice, maize, wheat, barley, millet, rye, sorghum or oats.

The activity of an EFTu polypeptide may be preferentially increased by increasing levels of the polypeptide (in a plastid). Alternatively, activity may also be increased when there is no change in levels of an EFTu, or even when there is a reduction in levels of an EFTu polypeptide. This may occur when the intrinsic properties of the polypeptide are altered, for example, by making mutant versions that are more active than the wild type polypeptide. Reference herein to “preferentially” increasing activity is taken to mean a targeted increase in activity in a plastid above that found in plastids of wild type plants under non-stressed conditions.

The activity may be preferentially increased in a plastid using techniques well known in the art, such as by targeting activity to the plastid using transit peptide sequences or by transformation of a plastid. Activity may be increased in any plastid, however, preferred is preferentially increasing activity in a chloroplast.

The term “EFTu (polypeptide) or a homologue thereof” as defined herein refers to a polypeptide comprising: (i) the following GTP-binding domains in any order GXXXXGK and DXXG and NKXD and S/L/K/Q A/G LN/F, where X is any amino acid; and (ii) EFTu domain ALMANPAIKR or a domain having in increasing order of preference at least 50%, 55%, 60%, 65%, 70%, 75%, 80%, 85%, 90% or 95% identity thereto and/or K D/G EE S/A.

An “EFTu polypeptide or a homologue thereof” may readily be identified using routine techniques well known in the art. The motifs defined above are highly conserved, thereby allowing a person skilled in the art to readily identify EFTu sequences falling within the definition above.

The plant EFTu polypeptide sequence represented by SEQ ID NO: 2, encoded by the nucleic acid of SEQ ID NO: 1, was found on the basis of homology to a transcription factor in Drosophila. Examples of plant-derived polypeptides falling under the definition of an “EFTu or a homologue thereof” include: SEQ ID NO: 4 from Nicotiana tabacum; SEQ ID NO: 6 from Arabidopsis thaliana; SEQ ID NO: 8 from Nicotiana sylvestris; SEQ ID NO: 10 a chloroplast translational elongation factor from rice; SEQ ID NO: 12 a mitochondrial elongation factor from Arabidopsis thaliana; SEQ ID NO: 14 a mitochondrial elongation factor from Arabidopsis thaliana; SEQ ID NO: 16 from Oryza sativa; SEQ ID NO: 18 from Zea mays (encoded by a nuclear gene for a mitochondrial product); SEQ ID NO: 20 from Arabidopsis thaliana; and SEQ ID NO: 22 Synechocystis.

The table below shows the percentage homology of various EFTu polypeptide sequences compared to the amino acid sequence represented by SEQ ID NO: 2 based on overall global sequence alignment. The percentage identity was calculated using an NCBI Align program with default parameters.

TABLE 1
Homology of EFTU-like protein sequences with SEQ ID NO: 2 based on
overall global sequence alignment

| | SEQ ID NO:/NCBI | Align full |
|---|---|---|
| EFTu Source and Type | Accession Number | length EMBOSS |
| Tobacco (EFTu B) | SEQ ID NO: 2 | |
| Tobacco (EFTu A) | SEQ ID NO: 4 | Identity: 439/489 |
| | M94204 | (89.8%) |
| | | # Similarity: 457/489 |
| | | (93.5%) |
| Tobacco (EFTu B) | SEQ ID NO: 8 | # Identity: 485/485 |
| | D11470 | (100.0%) |
| | | # Similarity: 485/485 |
| | | (100.0%) |
| Arabidopsis (EFTu A) | SEQ ID NO: 6 | Identity: 398/487 |
| | X52256 | (81.7%) |
| | | # Similarity: 434/487 |
| | | (89.1%) |
| Rice (EFTu A) | SEQ ID NO: 10 | |
| | AF145053 | |
| Arabidopsis (radicle up | S09152 | Identity: 398/487 |
| regulated EFTu) | | (81.7%) |
| | | # Similarity: 434/487 |
| | | (89.1%) |
| Rice (EFTu A) | AF327413 | Identity: 367/488 |
| | | (75.2%) |
| | | # Similarity: 411/488 |
| | | (84.2%) |
| Arabidopsis (tufA) | CAA36498.1 | |
| | At4g20360 | |
| | X52256 | |
| Arabidopsis (EFTu | SEQ ID NO: 12 | Identity: 297/494 |
| M-type - mitochondrial) | At4g02930 | (60.1%) |
| | | # Similarity: 346/494 |
| | | (70.0%) |
| Arabidopsis (EFTu | SEQ ID NO: 14 | # Identity: 295/507 |
| M-type - mitochondrial) | X89227 | (58.2%) |
| | (close to | # Similarity: 347/507 |
| | At4g02930) | (68.4%) |
| Rice (EFTu M-type - | SEQ ID NO: 16 | Identity: 287/500 |
| mitochondrial) | AF327062; | (57.4%) |
| | XM_470417 | # Similarity: 332/500 |
| | | (66.4%) |
| Arabidopsis EFTu M-type - | AL161495, X89227 | |
| mitochondrial) | | |
| Maize (EFTu M-type - | SEQ ID NO: 18 | Identity: 280/489 |
| mitochondrial) | AF264877 | (57.3%) |
| | | # Similarity: 333/489 |
| | | (68.1%) |
| Pea (EFTu) | CAA74893 | Identity: 405/495 |
| | | (81.8%) |
| | | # Similarity: 445/495 |
| | | (89.9%) |
| Soybean (tuf A) | CAA46864 | Identity: 409/492 |
| | | (83.1%) |
| | | # Similarity: 444/492 |
| | | (90.2%) |
| Tomato (EFTu) | BT014591 | Identity: 438/486 |
| | | (90.1%) |
| | | # Similarity: 456/486 |
| | | (93.8%) |
| Soybean (EFTu) | CAA61444 | Identity: 411/489 |
| | | (84.0%) |
| | | # Similarity: 440/489 |
| | | (90.0%) |
| Pelargonium (EFTu) | AAK08141 | Identity: 398/487 |
| | | (81.7%) |
| | | # Similarity: 428/487 |
| | | (87.9%) |

An assay may also be carried out to determine EFTu activity. A first step would involve isolating plastids (for example, chloroplasts), followed by obtaining purified EFTu for determination of the specific activity (GDP exchange). A person skilled in the art would readily be able to isolate plastids using techniques well known in the art. For an example of chloroplast isolation, see Olsson et al., (J Biol Chem. 2003 November 7:278(45): 44439-47). Similarly, a person skilled in the art would also readily be able to purify EFTu. As an example, see Stanzel et al., (Eur J Biochem. 1994 January 15:219(1-2):435-9) for a method for the purification of total EFTu. Furthermore, a person skilled in the art may also readily be able to determine the specific activity of EFTu. See for example Zhang et al., (J. Bacteriol. 176, 1184-1187) for a [³H]GDP exchange assay for the determination of the activity of recombinant pre-EFTu.

It is to be understood that sequences falling under the definition of “EFTu polypeptide or homologue thereof” are not to be limited to the sequences represented by SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20 and SEQ ID NO: 22, but that any polypeptide meeting the criteria of comprising motifs: (i) the following GTP-binding domains in any order GXXXXGK and DXXG and NKXD and S/L/K/Q A/G LN/F, where X is any amino acid; and (ii) EFTu domain ALMANPAIKR or a domain having 50% identity thereto and/or K D/G EE S/A may be suitable for use in the methods of the invention.

The nucleic acid encoding an EFTu polypeptide or a homologue thereof may be any natural or synthetic nucleic acid. An EFTu polypeptide or a homologue thereof as defined hereinabove is one that is encoded by an EFTu nucleic acid/gene. Therefore, the term “EFTu nucleic acid/gene” as defined herein is any nucleic acid/gene encoding an EFTu-like polypeptide or a homologue thereof as defined hereinabove. Examples of EFTu nucleic acids include those represented by any one of SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19 and SEQ ID NO: 21. EFTu nucleic acids/genes and variants thereof may be suitable in practising the methods of the invention. Variant EFTu nucleic acid/genes include portions of an EFTu nucleic acid/gene and/or nucleic acids capable of hybridising with an EFTu nucleic acid/gene.

The term portion as defined herein refers to a piece of DNA comprising at least enough nucleotides to encode a protein comprising: (i) the following GTP-binding domains in any order GXXXXGK and DXXG and NKXD and S/L/K/Q A/G LN/F, where X is any amino acid; and (ii) EFTu domain ALMANPAIKR or a domain having 50% identity thereto and/or K D/G EE S/A. A portion may be prepared, for example, by making one or more deletions to an EFTu nucleic acid. The portions may be used in isolated form or they may be fused to other coding (or non-coding) sequences in order to, for example, produce a protein that combines several activities. When fused to other coding sequences, the resulting polypeptide produced upon translation could be bigger than that predicted for the EFTu fragment. Preferably, the functional portion is a portion of a nucleic acid as represented by any one of SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19 and SEQ ID NO: 21.

Another variant EFTu nucleic acid/gene is a nucleic acid capable of hybridising under reduced stringency conditions, preferably under stringent conditions, with an EFTu nucleic acid/gene as hereinbefore defined, which hybridising sequence encodes a polypeptide comprising: (i) the following GTP-binding domains in any order GXXXXGK and DXXG and NKXD and S/L/K/Q A/G LN/F, where X is any amino acid; and (ii) EFTu domain ALMANPAIKR or a domain having 50% identity thereto and/or K D/G EE S/A. Preferably, the hybridising sequence is one that is capable of hybridising to a nucleic acid as represented by any one of SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19 and SEQ ID NO: 21 or to a portion of any of the aforementioned sequences as defined hereinabove.

The term “hybridisation” as defined herein is a process wherein substantially homologous complementary nucleotide sequences anneal to each other. The hybridisation process can occur entirely in solution, i.e. both complementary nucleic acids are in solution. The hybridisation process can also occur with one of the complementary nucleic acids immobilised to a matrix such as magnetic beads, Sepharose beads or any other resin. The hybridisation process can furthermore occur with one of the complementary nucleic acids immobilised to a solid support such as a nitro-cellulose or nylon membrane or immobilised by e.g. photolithography to, for example, a siliceous glass support (the latter known as nucleic acid arrays or microarrays or as nucleic acid chips). In order to allow hybridisation to occur, the nucleic acid molecules are generally thermally or chemically denatured to melt a double strand into two single strands and/or to remove hairpins or other secondary structures from single stranded nucleic acids. The stringency of hybridisation is influenced by conditions such as temperature, salt concentration, ionic strength and hybridisation buffer composition.

“Stringent hybridisation conditions” and “stringent hybridisation wash conditions” in the context of nucleic acid hybridisation experiments such as Southern and Northern hybridisations are sequence dependent and are different under different environmental parameters. The skilled artisan is aware of various parameters which may be altered during hybridisation and washing and which will either maintain or change the stringency conditions.

The Tₘ is the temperature under defined ionic strength and pH, at which 50% of the target sequence hybridises to a perfectly matched probe. The Tₘ is dependent upon the solution conditions and the base composition and length of the probe. For example, longer sequences hybridise specifically at higher temperatures. The maximum rate of hybridisation is obtained from about 16° C. up to 32° C. below Tₘ. The presence of monovalent cations in the hybridisation solution reduce the electrostatic repulsion between the two nucleic acid strands thereby promoting hybrid formation; this effect is visible for sodium concentrations of up to 0.4M. Formamide reduces the melting temperature of DNA-DNA and DNA-RNA duplexes with 0.6 to 0.7° C. for each percent formamide, and addition of 50% formamide allows hybridisaton to be performed at 30 to 45° C., though the rate of hybridisation will be lowered. Base pair mismatches reduce the hybridisation rate and the thermal stability of the duplexes. On average and for large probes, the Tₘ decreases about 1° C. per % base mismatch. The Tₘ may be calculated using the following equations, depending on the types of hybrids:

1. DNA-DNA hybrids (Meinkoth and Wahl, Anal. Biochem., 138: 267-284, 1984): Tₘ=81.5° C.+16.6×log[Na⁺]^{a} +0.41×% [G/C^{b}]−500×[L^{c}]⁻¹−0.61×% formamide
2. DNA-RNA or RNA-RNA hybrids: Tₘ=79.8+18.5 (log₁₀[Na⁺]^{a})+0.58 (% G/C^{b})+11.8 (% G/C^{b})²−820/L^{c}
3. oligo-DNA or oligo-RNA^{d} hybrids: For <20 nucleotides: Tₘ=2 (Iₙ) For 20-35 nucleotides: Tₘ=22+1.46 (Iₙ)
^{a} or for other monovalent cation, but only accurate in the 0.01-0.4 M range.
^{b} only accurate for % GC in the 30% to 75% range.
^{c} L=length of duplex in base pairs.
^{d} Oligo, oligonucleotide; Iₙ, effective length of primer=2×(no. of G/C)+(no. of A/T).

### Note: for each 1% formamide, the Tₘ is reduced by about 0.6 to 0.7° C., while the presence of 6 M urea reduces the Tₘ by about 30° C.

Specificity of hybridisation is typically the function of post-hybridisation washes. To remove background resulting from non-specific hybridisation, samples are washed with dilute salt solutions. Critical factors of such washes include the ionic strength and temperature of the final wash solution: the lower the salt concentration and the higher the wash temperature, the higher the stringency of the wash. Wash conditions are typically performed at or below hybridisation stringency. Generally, suitable stringent conditions for nucleic acid hybridisation assays or gene amplification detection procedures are as set forth above. Conditions of greater or less stringency may also be selected. Generally, low stringency conditions are selected to be about 50° C. lower than the thermal melting point (Tₘ) for the specific sequence at a defined ionic strength and pH. Medium stringency conditions are when the temperature is 20° C. below Tₘ, and high stringency conditions are when the temperature is 10° C. below Tₘ. For example, stringent conditions are those that are at least as stringent as, for example, conditions A-L; and reduced stringency conditions are at least as stringent as, for example, conditions M-R. Non-specific binding may be controlled using any one of a number of known techniques such as, for example, blocking the membrane with protein containing solutions, additions of heterologous RNA, DNA, and SDS to the hybridisation buffer, and treatment with Rnase. Examples of hybridisation and wash conditions are listed in Table 2 below.

TABLE 2
Examples of hybridisation and wash conditions

| | | | | Wash |
|---|---|---|---|---|
| Stringency | Polynucleotide | Hybrid | Hybridization Temperature | Temperature |
| Condition | Hybrid^{±} | Length (bp)^{‡} | and Buffer^{†} | and Buffer^{†} |
| A | DNA:DNA | > or | 65° C. 1xSSC; or 42° C., 1xSSC | 65° C.; |
| | | equal to 50 | and 50% formamide | 0.3xSSC |
| B | DNA:DNA | <50 | Tb*; 1xSSC | Tb*; 1xSSC |
| C | DNA:RNA | > or | 67° C. 1xSSC; or 45° C., 1xSSC | 67° C.; |
| | | equal to 50 | and 50% formamide | 0.3xSSC |
| D | DNA:RNA | <50 | Td*; 1xSSC | Td*; 1xSSC |
| E | RNA:RNA | > or | 70° C. 1xSSC; or 50° C., 1xSSC | 70° C.; |
| | | equal to 50 | and 50% formamide | 0.3xSSC |
| F | RNA:RNA | <50 | Tf*; 1xSSC | Tf*; 1xSSC |
| G | DNA:DNA | > or | 65° C. 4xSSC; or 45° C., 4xSSC | 65° C.; 1xSSC |
| | | equal to 50 | and 50% formamide | |
| H | DNA:DNA | <50 | Th*; 4 xSSC | Th*; 4xSSC |
| I | DNA:RNA | > or | 67° C. 4xSSC; or 45° C., 4xSSC | 67° C.; 1xSSC |
| | | equal to 50 | and 50% formamide | |
| J | DNA:RNA | <50 | Tj*; 4 xSSC | Tj*; 4xSSC |
| K | RNA:RNA | > or | 70° C. 4xSSC; or 40° C., 6xSSC | 67° C.; 1xSSC |
| | | equal to 50 | and 50% formamide | |
| L | RNA:RNA | <50 | Tl*; 2 xSSC | Tl*; 2xSSC |
| M | DNA:DNA | > or | 50° C. 4xSSC; or 40° C., 6xSSC | 50° C.; 2xSSC |
| | | equal to 50 | and 50% formamide | |
| N | DNA:DNA | <50 | Tn*; 6 xSSC | Tn*; 6xSSC |
| O | DNA:RNA | > or | 55° C. 4xSSC; or 42° C., 6xSSC | 55° C.; 2xSSC |
| | | equal to 50 | and 50% formamide | |
| P | DNA:RNA | <50 | Tp*; 6 xSSC | Tp*; 6xSSC |
| Q | RNA:RNA | > or | 60° C. 4xSSC; or 45° C., 6xSSC | 60° C.; |
| | | equal to 50 | and 50% formamide | 2xSSC |
| R | RNA:RNA | <50 | Tr*; 4 xSSC | Tr*; 4xSSC |

^{‡}The “hybrid length” is the anticipated length for the hybridising nucleic acid. When nucleic acids of known sequence are hybridised, the hybrid length may be determined by aligning the sequences and identifying the conserved regions described herein.
^{†}SSPE (1xSSPE is 0.15M NaCl, 10 mM NaH₂PO₄, and 1.25 mM EDTA, pH 7.4) may be substituted for SSC (1xSSC is 0.15M NaCl and 15 mM sodium citrate) in the hybridisation and wash buffers; washes are performed for 15 minutes after hybridisation is complete. The hybridisations and washes may additionally include 5 × Denhardt's reagent, .5-1.0% SDS, 100 μg/ml denatured, fragmented salmon sperm DNA, 0.5% sodium pyrophosphate, and up to 50% formamide.
*Tb − Tr: The hybridisation temperature for hybrids anticipated to be less than 50 base pairs in length should be 5-10° C. less than the melting temperature Tₘ of the hybrids; the Tₘ is determined according to the above-mentioned equations.
^{±}The present invention also encompasses the substitution of any one, or more DNA or RNA hybrid partners with either a PNA, or a modified nucleic acid.

For the purposes of defining the level of stringency, reference can be made to Sambrook et al. (2001) Molecular Cloning: a laboratory manual, 3^{rd} Edition Cold Spring Harbor Laboratory Press, CSH, New York or to Current Protocols in Molecular Biology, John Wiley & Sons, N.Y. (1989).

The EFTu nucleic acid or variant thereof may be derived from any natural or artificial source. The nucleic acid/gene or variant thereof may be isolated from a microbial source, such as bacteria, yeast or fungi, or from a plant, algae or animal source. This nucleic acid may be modified from its native form in composition and/or genomic environment through deliberate human manipulation. The nucleic acid is preferably of plant origin, whether from the same plant species (for example to the one in which it is to be introduced) or whether from a different plant species. The nucleic acid may be isolated from a dicotyledonous plant, preferably from the family Solanaceae, further preferably from the genus Nicotianae, more preferably from tobacco. Most preferably, the EFTu nucleic acid isolated from tobacco is represented by SEQ ID NO: 1 and the EFTu amino acid sequence is as represented by SEQ ID NO: 2. The nucleic acid of plant origin is preferably a plastidic nucleic acid (i.e. derived from a plastid). The plastidic nucleic acids have a closer relationship to bacterial nucleic acids than to non-plastidic nucleic acids. Therefore, the invention may also be performed using bacterial EFTu nucleic acids or variants thereof. The bacterial nucleic acids are targeted to a plastid, preferably to a chloroplast. Furthermore, mitochondria also share a common origin to the plastidic and bacterial nucleic acids and therefore the invention may also be performed using a mitochondrial EFTu nucleic acid or variant thereof which may be from animal or fungal origin. The mitochondrial nucleic acids are targeted to a plastid, preferably to a chloroplast. Despite being less closely related to a plastidic nucleic acid than bacterial or mitochondrial nucleic acid, a cytosolic nucleic acid may also be suitable for use in the methods of the invention so long as the nucleic acid is targeted to a plastid, preferably to a chloroplast.

Methods for targeting to plastids are well known in the art and include the use of transit peptides. Table 3 below shows examples of transit peptides which can be used to target any EFTu protein to a plastid, which EFTu is not, in its natural form, normally targeted to a plastid, or which EFTu in its natural form is targeted to a plastid by virtue of a different transit peptide (for example, its natural transit peptide).

TABLE 3

Examples of transit peptide sequences useful in
targeting amino acids to plastids

| NCBI | | | |
|---|---|---|---|
| Accession | | | |
| Number/ | Source | Protein | |
| SEQ ID NO | Organism | Function | Transit Peptide Sequence |
| SEQ ID NO: | Chlamydomonas | Ferredoxin | MAMAMRSTFAARVGAKPAVRGARPASRMSCMA |
| P07839 | | | |
| SEQ ID NO: | Chlamydomonas | Rubisco | MQVTMKSSAVSGQRVGGARVATRSVRRAQLQV |
| AAR23425 | | activase | |
| SEQ ID NO: | Arabidopsis | asp Amino | MASLMLSLGSTSLLPREINKDKLKLGTSASNPFLKAKSFSRVT |
| CAA56932 | thaliana | transferase | MTVAVKPSR |
| SEQ ID NO: | Arabidopsis | Acyl carrier | MATQFSASVSLQTSCLATTRISFQKPALISNHGKTNLSFNLRR |
| CAA31991 | thaliana | protein1 | SIPSRRLSVSC |
| SEQ ID NO: | Arabidopsis | Acyl carrier | MASIAASASISLQARPRQLAIAASQVKSFSNGRRSSLSFNLRQ |
| CAB63798 | thaliana | protein2 | LPTRLTVSCAAKPETVDKVCAVVRKQL |
| SEQ ID NO: | Arabidopsis | Acyl carrier | MASIATSASTSLQARPRQLVIGAKQVKSFSYGSRSNLSFNLR |
| CAB63799 | thaliana | protein3 | QLPTRLTVYCAAKPETVDKVCAVVRKQLSLKE |

The activity of an EFTu polypeptide or a homologue thereof may be increased by introducing a genetic modification (preferably in the locus of an EFTu gene). The locus of a gene as defined herein is taken to mean a genomic region, which includes the gene of interest and 10 KB up- or downstream of the coding region.

The genetic modification may be introduced, for example, by any one (or more) of the following methods: TDNA activation, TILLING, site-directed mutagenesis, directed evolution, homologous recombination and by introducing and expressing in a plant cell a nucleic acid encoding an EFTu polypeptide or a homologue thereof (the gene product may then be targeted to a plastid in the plant cell, unless the gene being expressed is a plastidic gene). Following introduction of the genetic modification, there follows a step of selecting for increased activity of an EFTu polypeptide, which increase in activity gives plants having increased yield.

T-DNA activation tagging (Hayashi et al. Science (1992) 1350-1353) involves insertion of T-DNA usually containing a promoter (may also be a translation enhancer or an intron), in the genomic region of the gene of interest or 10 KB up- or down stream of the coding region of a gene in a configuration such that the promoter directs expression of the targeted gene. Typically, regulation of expression of the targeted gene by its natural promoter is disrupted and the gene falls under the control of the newly introduced promoter. The promoter is typically embedded in a T-DNA. This T-DNA is randomly inserted into the plant genome, for example, through Agrobacterium infection and leads to overexpression of genes near to the inserted T-DNA. The resulting transgenic plants show dominant phenotypes due to overexpression of genes close to the introduced promoter. The promoter to be introduced may be any promoter capable of directing expression of a gene in the desired organism, in this case a plant. For example, constitutive, tissue-preferred, cell type-preferred and inducible promoters are all suitable for use in T-DNA activation. Preferred is the use of a seed-specific promoter, more particularly an embryo- and/or alerone-specific promoter.

A genetic modification may also be introduced in the locus of an EFTu gene using the technique of TILLING (Targeted Induced Local Lesions IN Genomes). This is a mutagenesis technology useful to generate and/or identify, and to eventually isolate mutagenised variants of an EFTu nucleic acid capable of exhibiting EFTu activity. TILLING also allows selection of plants carrying such mutant variants. These mutant variants may even exhibit higher EFTu activity than that exhibited by the gene in its natural form. TILLNG combines high-density mutagenesis with high-throughput screening methods. The steps typically followed in TILLING are: (a) EMS mutagenesis (Redei and Koncz, 1992; Feldmann et al., 1994; Lightner and Caspar, 1998); (b) DNA preparation and pooling of individuals; (c) PCR amplification of a region of interest; (d) denaturation and annealing to allow formation of heteroduplexes; (e) DHPLC, where the presence of a heteroduplex in a pool is detected as an extra peak in the chromatogram; (f) identification of the mutant individual; and (g) sequencing of the mutant PCR product. Methods for TILLING are well known in the art (McCallum Nat Biotechnol. 2000 April; 18(4):455-7, reviewed by Stemple 2004 (TILLING-a high-throughput harvest for functional genomics. Nat. Rev. Genet. 2004 February; 5(2):145-50.)).

Site-directed mutagenesis may be used to generate variants of EFTu nucleic acids. Several methods are available to achieve site-directed mutagenesis, the most common being PCR-based methods (current protocols in molecular biology. Wiley Eds. http://www.4ulr.com/products/currentprotocols/index.html).

Directed evolution (or gene shuffling) may also be used to generate variants of EFTu nucleic acids. This consists of iterations of DNA shuffling followed by appropriate screening and/or selection to generate and identify variants having an modified biological activity (Castle et al., (2004) Science 304(5674): 1151-4; U.S. Pat. Nos. 5,811,238 and 6,395,547).

TDNA activation, TILLING, directed evolution and site-directed mutagenesis are examples of technologies that enable the generation of novel alleles and EFTu variants.

Homologous recombination allows introduction in a genome of a selected nucleic acid at a defined selected position. Homologous recombination is a standard technology used routinely in biological sciences for lower organisms such as yeast or the moss physcomitrella. Methods for performing homologous recombination in plants have been described not only for model plants (Offringa et al. Extrachromosomal homologous recombination and gene targeting in plant cells after Agrobacterium-mediated transformation. 1990 EMBO J. 1990 October; 9(10):3077-84) but also for crop plants, for example rice (Terada R, Urawa H, Inagaki Y, Tsugane K, Iida S. Efficient gene targeting by homologous recombination in rice. Nat Biotechnol. 2002. Iida and Terada: A tale of two integrations, transgene and T-DNA: gene targeting by homologous recombination in rice. Curr Opin Biotechnol. 2004 April; 15(2):132-8). The nucleic acid to be targeted (which may be an EFTu nucleic acid or variant thereof as hereinbefore defined) need not be targeted to the locus of an EFTu gene, but may be introduced in, for example, regions of high expression. The nucleic acid to be targeted may be an improved allele used to replace the endogenous gene or may be introduced in addition to the endogenous gene.

According to a preferred embodiment of the invention, plant yield may be increased in plants grown under non-stressed conditions by introducing and expressing in a plant, plant part or plant cell a nucleic acid encoding an EFTu polypeptide or a homologue thereof. The polypeptide may then be targeted to a plastid in the plant cell, unless the gene being expressed is a plastidic gene. An EFTu polypeptide or a homologue thereof as mentioned above is one comprising: (i) the following GTP-binding domains in any order GXXXXGK and DXXG and NKXD and S/L/K/Q A/G LN/F, where X is any amino acid; and (ii) EFTu domain ALMANPAIKR or a domain having 50% identity thereto and/or K D/G EE S/A. The nucleic acid to be introduced into a plant may be a full-length nucleic acid or may be a portion or a hybridizing sequence as hereinbefore defined.

“Homologues” of a protein encompass peptides, oligopeptides, polypeptides, proteins and enzymes having amino acid substitutions, deletions and/or insertions relative to the unmodified protein in question and having similar biological and functional activity as the unmodified protein from which they are derived. To produce such homologues, amino acids of the protein may be replaced by other amino acids having similar properties (such as similar hydrophobicity, hydrophilicity, antigenicity, propensity to form or break α-helical structures or β-sheet structures). Conservative substitution tables are well known in the art (see for example Creighton (1984) Proteins. W. H. Freeman and Company and Table 1 above).

According to a preferred feature of the invention, the homologue has in increasing order of preference at least 45%, 50%, 55%, 60%, 65%, 70%, 75%, 80%, 85%, 90% or 95% sequence identity to the amino acid sequence represented by SEQ ID NO: 2. Whether a polypeptide has at least 45%, 50%, 55%, 60%, 65%, 70%, 75%, 80%, 85%, 90% or 95% identity to the amino acid represented by SEQ ID NO: 2 may readily be established by sequence alignment. Methods for the alignment of sequences for comparison are well known in the art, such methods include GAP, BESTFIT, BLAST, FASTA and TFASTA. GAP uses the algorithm of Needleman and Wunsch (J. Mol. Biol. 48: 443-453, 1970) to find the alignment of two complete sequences that maximises the number of matches and minimises the number of gaps. The BLAST algorithm calculates percent sequence identity and performs a statistical analysis of the similarity between the two sequences. The software for performing BLAST analysis is publicly available through the National Centre for Biotechnology Information. An EFTu polypeptide or a homologue thereof having at least 45%, 50%, 55%, 60%, 65%, 70%, 75%, 80%, 85%, 90% or 95% identity to the amino acid represented by SEQ ID NO: 2 may readily be identified by aligning a query sequence (preferably a protein sequence) with known EFTu protein sequences (see for example the alignment shown in FIG. 1). The query sequence may be aligned (with known EFTU-like sequences) using, for example, the VNTI AlignX multiple alignment program, based on a modified clustal W algorithm (InforMax, Bethesda, MD, http://www.informaxinc.com), with default settings for gap opening penalty of 10 and a gap extension of 0.05.

Also encompassed by the term “homologues” are two special forms of homology, which include orthologous sequences and paralogous sequences, which encompass evolutionary concepts used to describe ancestral relationships of genes. The term “paralogous” relates to gene-duplications within the genome of a species leading to paralogous genes. The term “orthologous” relates to homologous genes in different organisms due to speciation.

Othologues in, for example, monocot plant species may easily be found by performing a so-called reciprocal blast search. This may be done by a first blast involving blasting a query sequence (for example, SEQ ID NO: 1 or SEQ ID NO: 2) against any sequence database, such as the publicly available NCBI database which may be found at: http://www.ncbi.nim.nih.gov. BLASTN or TBLASTX (using standard default values) may be used when starting from a nucleotide sequence and BLASTP or TBLASTN (using standard default values) may be used when starting from a protein sequence. The BLAST results may optionally be filtered. The full-length sequences of either the filtered results or non-filtered results are then BLASTed back (second BLAST) against sequences from the organism from which the query sequence is derived (where the query sequence is SEQ ID NO: 1 or SEQ ID NO: 2 the second blast would therefore be against tobacco sequences). The results of the first and second BLASTs are then compared. A paralogue is identified if a high-ranking hit from the second blast is from the same species as from which the query sequence is derived; an orthologue is identified if a high-ranking hit is not from the same species as from which the query sequence is derived. High-ranking hits are those having a low E-value. The lower the E-value, the more significant the score (or in other words the lower the chance that the hit was found by chance). Computation of the E-value is well known in the art. In the case of large families, ClustalW may be used, followed by a neighbour joining tree, to help visualize clustering of related genes and to identify orthologues and paralogues.

A homologue may be in the form of a “substitutional variant” of a protein, i.e. where at least one residue in an amino acid sequence has been removed and a different residue inserted in its place. Amino acid substitutions are typically of single residues, but may be clustered depending upon functional constraints placed upon the polypeptide; insertions will usually be of the order of about 1 to 10 amino acid residues. Preferably, amino acid substitutions comprise conservative amino acid substitutions.

A homologue may also be in the form of an “insertional variant” of a protein, i.e. where one or more amino acid residues are introduced into a predetermined site in a protein. Insertions may comprise amino-terminal and/or carboxy-terminal fusions as well as intra-sequence insertions of single or multiple amino acids. Generally, insertions within the amino acid sequence will be smaller than amino- or carboxy-terminal fusions, of the order of about 1 to 10 residues. Examples of amino- or carboxy-terminal fusion proteins or peptides include the binding domain or activation domain of a transcriptional activator as used in the yeast two-hybrid system, phage coat proteins, (histidine)6-tag, glutathione S-transferase-tag, protein A, maltose-binding protein, dihydrofolate reductase, Tag-100 epitope, c-myc epitope, FLAG®-epitope, lacZ, CMP (calmodulin-binding peptide), HA epitope, protein C epitope and VSV epitope.

Homologues in the form of “deletion variants” of a protein are characterised by the removal of one or more amino acids from a protein.

Amino acid variants of a protein may readily be made using peptide synthetic techniques well known in the art, such as solid phase peptide synthesis and the like, or by recombinant DNA manipulations. Methods for the manipulation of DNA sequences to produce substitution, insertion or deletion variants of a protein are well known in the art. For example, techniques for making substitution mutations at predetermined sites in DNA are well known to those skilled in the art and include M13 mutagenesis, T7-Gen in vitro mutagenesis (USB, Cleveland, Ohio), QuickChange Site Directed mutagenesis (Stratagene, San Diego, Calif.), PCR-mediated site-directed mutagenesis or other site-directed mutagenesis protocols. The EFTu polypeptide or homologue thereof may be a derivative. “Derivatives” include peptides, oligopeptides, polypeptides, proteins and enzymes which may comprise substitutions, deletions or additions of naturally and non-naturally occurring amino acid residues compared to the amino acid sequence of a naturally-occurring form of the protein, for example, as presented in SEQ ID NO: 2. “Derivatives” of a protein encompass peptides, oligopeptides, polypeptides, proteins and enzymes which may comprise naturally occurring altered, glycosylated, acylated or non-naturally occurring amino acid residues compared to the amino acid sequence of a naturally-occurring form of the polypeptide. A derivative may also comprise one or more non-amino acid substituents compared to the amino acid sequence from which it is derived, for example a reporter molecule or other ligand, covalently or non-covalently bound to the amino acid sequence, such as a reporter molecule which is bound to facilitate its detection, and non-naturally occurring amino acid residues relative to the amino acid sequence of a naturally-occurring protein.

The EFTu polypeptide or homologue thereof may be encoded by an alternative splice variant of an EFTu nucleic acid/gene. The term “alternative splice variant” as used herein encompasses variants of a nucleic acid sequence in which selected introns and/or exons have been excised, replaced or added. Such variants will be ones in which the biological activity of the protein is retained, which may be achieved by selectively retaining functional segments of the protein. Such splice variants may be found in nature or may be manmade. Methods for making such splice variants are well known in the art. Preferred splice variants are splice variants of the nucleic acid represented by any one of SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19 and SEQ ID NO: 21. Splice variants useful in the methods of the invention are those encoding polypeptides comprising: (i) the following GTP-binding domains in any order GXXXXGK and DXXG and NKXD and S/L/K/Q A/G LN/F, where X is any amino acid; and (ii) EFTu domain ALMANPAIKR or a domain having 50% identity thereto and/or K D/G EE S/A.

The homologue may also be encoded by an allelic variant of a nucleic acid encoding an EFTu polypeptide or a homologue thereof, preferably an allelic variant of the nucleic acid represented by SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19 and SEQ ID NO: 21. Useful in the methods of the invention are allelic variants encoding polypeptides which comprise: (i) the following GTP-binding domains in any order GXXXXGK and DXXG and NKXD and S/L/K/Q A/G LN/F, where X is any amino acid; and (ii) EFTu domain ALMANPAIKR or a domain having 50% identity thereto and/or K D/G EE S/A. Allelic variants exist in nature and encompassed within the methods of the present invention is the use of these natural alleles. Allelic variants encompass Single Nucleotide Polymorphisms (SNPs), as well as Small Insertion/Deletion Polymorphisms (INDELs). The size of INDELs is usually less than 100 bp. SNPs and INDELs form the largest set of sequence variants in naturally occurring polymorphic strains of most organisms.

According to the present invention, enhanced or increased expression of the EFTu nucleic acid or variant thereof is envisaged. Methods for obtaining enhanced or increased expression of genes or gene products are well documented in the art and include, for example, overexpression driven by appropriate promoters, the use of transcription enhancers or translation enhancers. Isolated nucleic acids which serve as promoter or enhancer elements may be introduced in an appropriate position (typically upstream) of a non-heterologous form of a polynucleotide so as to upregulate expression of an EFTu nucleic acid or variant thereof. For example, endogenous promoters may be altered in vivo by mutation, deletion, and/or substitution (see, Kmiec, U.S. Pat. No. 5,565,350; Zarling et al., PCT/US93/03868), or isolated promoters may be introduced into a plant cell in the proper orientation and distance from a gene of the present invention so as to control the expression of the gene.

If polypeptide expression is desired, it is generally desirable to include a polyadenylation region at the 3′-end of a polynucleotide coding region. The polyadenylation region can be derived from the natural gene, from a variety of other plant genes, or from T-DNA. The 3′ end sequence to be added may be derived from, for example, the nopaline synthase or octopine synthase genes, or alternatively from another plant gene, or less preferably from any other eukaryotic gene. Methods for preferentially increasing activity of an EFTu polypeptide in a plastid are described hereinabove.

An intron sequence may also be added to the 5′ untranslated region or the coding sequence of the partial coding sequence to increase the amount of the mature message that accumulates in the cytosol. Inclusion of a spliceable intron in the transcription unit in both plant and animal expression constructs has been shown to increase gene expression at both the mRNA and protein levels up to 1000-fold, Buchman and Berg, Mol. Cell biol. 8:4395-4405 (1988); Callis et al., Genes Dev. 1:1183-1200 (1987). Such intron enhancement of gene expression is typically greatest when placed near the 5′ end of the transcription unit. Use of the maize introns Adh1-S intron 1, 2, and 6, the Bronze-1 intron are known in the art. See generally, The Maize Handbook, Chapter 116, Freeling and Walbot, Eds., Springer, N.Y. (1994).

The invention also provides genetic constructs and vectors to facilitate introduction and/or expression of the nucleotide sequences useful in the methods according to the invention.

Therefore, there is provided a gene construct comprising:
(i) An EFTu nucleic acid or variant thereof encoding a polypeptide comprising: (a) the following GTP-binding domains in any order GXXXXGK and DXXG and NKXD and S/L/K/Q A/G LN/F, where X is any amino acid; and (b) EFTu domain ALMANPAIKR or a domain having 50% identity thereto and/or K D/G EE S/A; and
(ii) One or more control sequences capable of driving expression of the nucleic acid sequence of (i), preferably wherein the one or more control sequences comprise at least a seed-specific promoter; and optionally
(iii) A transcription termination sequence.

Constructs useful in the methods according to the present invention may be constructed using recombinant DNA technology well known to persons skilled in the art. The gene constructs may be inserted into vectors, which may be commercially available, suitable for transforming into plants and suitable for expression of the gene of interest in the transformed cells. Plants are transformed with a vector comprising the sequence of interest (i.e., an EFTu nucleic acid or variant thereof). The sequence of interest is operably linked to one or more control sequences (at least to a promoter). The terms “regulatory element”, “control sequence” and “promoter” are all used interchangeably herein and are to be taken in a broad context to refer to regulatory nucleic acid sequences capable of effecting expression of the sequences to which they are ligated. Encompassed by the aforementioned terms are transcriptional regulatory sequences derived from a classical eukaryotic genomic gene (including the TATA box which is required for accurate transcription initiation, with or without a CCAAT box sequence) and additional regulatory elements (i.e. upstream activating sequences, enhancers and silencers) which alter gene expression in response to developmental and/or external stimuli, or in a tissue-specific manner. Also included within the term is a transcriptional regulatory sequence of a classical prokaryotic gene, in which case it may include a −35 box sequence and/or −10 box transcriptional regulatory sequences. The term “regulatory element” also encompasses a synthetic fusion molecule or derivative which confers, activates or enhances expression of a nucleic acid molecule in a cell, tissue or organ. The term “operably linked” as used herein refers to a functional linkage between the promoter sequence and the gene of interest, such that the promoter sequence is able to initiate transcription of the gene of interest.

Advantageously, any type of promoter may be used to drive expression of the nucleic acid sequence, however our studies have revealed that some promoters outperform others in the methods of the invention. The promoter is preferrably a tissue-specific promoter, i.e. one that is capable of predominantly initiating transcription in certain tissues, such as the leaves, roots, seed tissue etc., substantially to the exclusion of initiating transcription elsewhere in the plant, but whilst still allowing for residual expression in other parts of a plant due to leaky promoters.

Our studies have revealed that use of seed-specific promoters, more particularly embryo-specific and/or alerone-specific promoters, perform better in the methods of the invention (i.e. give plants having better yield) than constitutive promoters in the same methods. It is therefore preferred that the EFTu nucleic acid or variant thereof is operably linked to a seed-specific promoter. A seed-specific promoter is transcriptionally active predominantly in seed tissue, but not necessarily exclusively in seed tissue (in case of leaky expression). Seed-specific promoters are well known in the art. Preferably, the seed-specific promoter is an embryo-specific and/or aleurone-specific promoter, more preferably an oleosin promoter (see for example, SEQ ID NO: 29 which represents the sequence of the oleosin promoter from rice). It should be clear that the applicability of the present invention is not restricted to the EFTu-like nucleic acid represented by SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19 and SEQ ID NO: 21, nor is the applicability of the invention restricted to expression of an EFTu nucleic acid when driven by an oleosin promoter.

Optionally, one or more terminator sequences may also be used in the construct introduced into a plant. The term “terminator” encompasses a control sequence which is a DNA sequence at the end of a transcriptional unit which signals 3′ processing and polyadenylation of a primary transcript and termination of transcription. Additional regulatory elements may include transcriptional as well as translational enhancers. Those skilled in the art will be aware of terminator and enhancer sequences which may be suitable for use in performing the invention. Such sequences would be known or may readily be obtained by a person skilled in the art.

The genetic constructs of the invention may further include an origin of replication sequence which is required for maintenance and/or replication in a specific cell type. One example is when a genetic construct is required to be maintained in a bacterial cell as an episomal genetic element (e.g. plasmid or cosmid molecule). Preferred origins of replication include, but are not limited to, the f1-ori and colE1.

The genetic construct may optionally comprise a selectable marker gene. As used herein, the term “selectable marker gene” includes any gene that confers a phenotype on a cell in which it is expressed to facilitate the identification and/or selection of cells that are transfected or transformed with a nucleic acid construct of the invention. Suitable markers may be selected from markers that confer antibiotic or herbicide resistance, that introduce a new metabolic trait or that allow visual selection. Examples of selectable marker genes include genes conferring resistance to antibiotics (such as nptil that phosphorylates neomycin and kanamycin, or hpt, phosphorylating hygromycin), to herbicides (for example bar which provides resistance to Basta; aroA or gox providing resistance against glyphosate), or genes that provide a metabolic trait (such as manA that allows plants to use mannose as sole carbon source). Expression of visual marker genes results in the formation of colour (for example β-glucuronidase, GUS), luminescence (such as luciferase) or fluorescence (Green Fluorescent Protein, GFP, and derivatives thereof).

The present invention also encompasses plants and plant parts obtainable by the methods according to the present invention. The present invention therefore provides plants or plant parts obtainable by the methods according to the present invention, which plants or plant parts comprise an EFTu nucleic acid or variant thereof (transgene).

The invention also provides a method for the production of transgenic plants having increased yield under non-stressed conditions, comprising introduction and expression in a plant or plant part (including plant cell) of an EFTu nucleic acid or a variant thereof. The gene product of expression of the nucleic acid is targeted to a plastid in a plant cell if it is not already in the plastid.

More specifically, the present invention provides a method for the production of transgenic plants having increased yield under non-stressed conditions, which method comprises:
(i) introducing and expressing in a plant or plant part (including plant cell) an EFTu nucleic acid or variant thereof encoding a polypeptide comprising: (a) the following GTP-binding domains in any order GXXXXGK and DXXG and NKXD and S/L/K/Q A/G LN/F, where X is any amino acid; and (b) EFTu domain ALMANPAIKR or a domain having 50% identity thereto and/or K D/G EE S/A, which the polypeptide is targeted to a plastid in a plant cell if it is not already in the plastid; and
(ii) cultivating the plant or plant part under non-stress growth conditions promoting plant growth and development.

The nucleic acid may be introduced directly into a plant cell or into the plant itself (including introduction into a tissue (such as a plastid), organ or any other part of a plant). According to a preferred feature of the present invention, the nucleic acid is preferably introduced into a plant by transformation.

The term “transformation” as referred to herein encompasses the transfer of an exogenous polynucleotide into a host cell, irrespective of the method used for transfer. Plant tissue capable of subsequent clonal propagation, whether by organogenesis or embryogenesis, may be transformed with a genetic construct of the present invention and a whole plant regenerated therefrom. The particular tissue chosen will vary depending on the clonal propagation systems available for, and best suited to, the particular species being transformed. Exemplary tissue targets include leaf disks, pollen, embryos, cotyledons, hypocotyls, megagametophytes, callus tissue, existing meristematic tissue (e.g., apical meristem, axillary buds, and root meristems), and induced meristem tissue (e.g., cotyledon meristem and hypocotyl meristem). The polynucleotide may be transiently or stably introduced into a host cell and may be maintained non-integrated, for example, as a plasmid. Alternatively, it may be integrated into the host genome. The resulting transformed plant cell may then be used to regenerate a transformed plant in a manner known to persons skilled in the art.

Transformation of plant species is now a fairly routine technique. Advantageously, any of several transformation methods may be used to introduce the gene of interest into a suitable ancestor cell. Transformation methods include the use of liposomes, electroporation, chemicals that increase free DNA uptake, injection of the DNA directly into the plant, particle gun bombardment, transformation using viruses or pollen and microprojection. Methods may be selected from the calcium/polyethylene glycol method for protoplasts (Krens, F. A. et al., 1882, Nature 296, 72-74; Negrutiu I. et al., June 1987, Plant Mol. Biol. 8, 363-373); electroporation of protoplasts (Shillito R. D. et al., 1985 Bio/Technol 3, 1099-1102); microinjection into plant material (Crossway A. et al., 1986, Mol. Gen Genet 202, 179-185); DNA or RNA-coated particle bombardment (Klein T. M. et al., 1987, Nature 327, 70) infection with (non-integrative) viruses and the like. Transgenic rice plants expressing an EFTu nucleic acid/gene are preferably produced via Agrobacterium-mediated transformation using any of the well known methods for rice transformation, such as described in any of the following: published European patent application EP 1198985 A1, Aldemita and Hodges (Planta, 199, 612-617, 1996); Chan et al. (Plant Mol. Biol. 22 (3) 491-506, 1993), Hiei et al. (Plant J. 6 (2) 271-282, 1994), which disclosures are incorporated by reference herein as if fully set forth. In the case of corn transformation, the preferred method is as described in either Ishida et al. (Nat. Biotechnol. 1996 June; 14(6): 745-50) or Frame et al. (Plant Physiol. 2002 May; 129(1): 13-22), which disclosures are incorporated by reference herein as if fully set forth.

Generally after transformation, plant cells or cell groupings are selected for the presence of one or more markers which are encoded by plant-expressible genes co-transferred with the gene of interest, following which the transformed material is regenerated into a whole plant.

Following DNA transfer and regeneration, putatively transformed plants may be evaluated, for instance using Southern analysis, for the presence of the gene of interest, copy number and/or genomic organisation. Alternatively or additionally, expression levels of the newly introduced DNA may be monitored using Northern and/or Western analysis, both techniques being well known to persons having ordinary skill in the art.

The generated transformed plants may be propagated by a variety of means, such as by clonal propagation or classical breeding techniques. For example, a first generation (or T1) transformed plant may be selfed to give homozygous second generation (or T2) transformants, and the T2 plants further propagated through classical breeding techniques.

The generated transformed organisms may take a variety of forms. For example, they may be chimeras of transformed cells and non-transformed cells; clonal transformants (e.g., all cells transformed to contain the expression cassette); grafts of transformed and untransformed tissues (e.g., in plants, a transformed rootstock grafted to an untransformed scion).

The present invention clearly extends to any plant cell or plant produced by any of the methods described herein, and to all plant parts and propagules thereof. The present invention extends further to encompass the progeny of a primary transformed or transfected cell, tissue, organ or whole plant that has been produced by any of the aforementioned methods, the only requirement being that progeny exhibit the same genotypic and/or phenotypic characteristic(s) as those produced in the parent by the methods according to the invention. The invention also includes host cells containing an isolated EFTu nucleic acid or variant thereof. Preferred host cells according to the invention are plant cells.

The invention also extends to harvestable parts of a plant, such as but not limited to seeds, leaves, fruits, flowers, stem cultures, rhizomes, tubers and bulbs. The invention further relates to products derived (preferably directly) from a harvestable part of such a plant, such products including dry pellets or powders, oil, fat and fatty acids, starch or proteins.

The present invention also encompasses use of EFTu nucleic acids or variants thereof and to the use of EFTu polypeptides or homologues thereof in increasing yield, especially seed yield, in plants grown under non-stress conditions. The seed yield is as defined hereinabove.

EFTu nucleic acids or variants thereof, or EFTu polypeptides or homologues thereof may find use in breeding programmes in which a DNA marker is identified which may be genetically linked to an EFTu gene or variant thereof. The EFTu nucleic acids/ genes or variants thereof, or EFTu polypeptides or homologues thereof may be used to define a molecular marker. This DNA or protein marker may then be used in breeding programs to select plants having increased yield. The EFTu gene or variant thereof may, for example, be a nucleic acid as represented by any one of SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19 and SEQ ID NO: 21.

Allelic variants of an EFTu nucleic acid/gene may also find use in marker-assisted breeding programmes. Such breeding programmes sometimes require introduction of allelic variation by mutagenic treatment of the plants, using for example EMS mutagenesis; alternatively, the programme may start with a collection of allelic variants of so called “natural” origin caused unintentionally. Identification of allelic variants then takes place by, for example, PCR. This is followed by a selection step for selection of superior allelic variants of the sequence in question and which give increased plant yield. Selection is typically carried out by monitoring growth performance of plants containing different allelic variants of the sequence in question, for example, different allelic variants of any one of SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19 and SEQ ID NO: 21. Growth performance may be monitored in a greenhouse or in the field. Further optional steps include crossing plants, in which the superior allelic variant was identified, with another plant. This could be used, for example, to make a combination of interesting phenotypic features.

An EFTu nucleic acid or variant thereof may also be used as probes for genetically and physically mapping the genes that they are a part of, and as markers for traits linked to those genes. Such information may be useful in plant breeding in order to develop lines with desired phenotypes. Such use of EFTu nucleic acids or variants thereof requires only a nucleic acid sequence of at least 15 nucleotides in length. The EFTu nucleic acids or variants thereof may be used as restriction fragment length polymorphism (RFLP) markers. Southern blots (Maniatis) of restriction-digested plant genomic DNA may be probed with the EFTu nucleic acids or variants thereof. The resulting banding patterns may then be subjected to genetic analyses using computer programs such as MapMaker (Lander et al. (1987) Genomics 1:174-181) in order to construct a genetic map. In addition, the nucleic acids may be used to probe Southern blots containing restriction endonuclease-treated genomic DNAs of a set of individuals representing parent and progeny of a defined genetic cross. Segregation of the DNA polymorphisms is noted and used to calculate the position of the EFTu nucleic acid or variant thereof in the genetic map previously obtained using this population (Botstein et al. (1980) Am. J. Hum. Genet. 32:314-331).

The production and use of plant gene-derived probes for use in genetic mapping is described in Bematzky and Tanksley (1986) Plant Mol. Biol. Reporter 4:37-41. Numerous publications describe genetic mapping of specific cDNA clones using the methodology outlined above or variations thereof. For example, F2 intercross populations, backcross populations, randomly mated populations, near isogenic lines, and other sets of individuals may be used for mapping. Such methodologies are well known to those skilled in the art.

The nucleic acid probes may also be used for physical mapping (i.e., placement of sequences on physical maps; see Hoheisel et al. In: Non-mammalian Genomic Analysis: A Practical Guide, Academic press 1996, pp. 319-346, and references cited therein).

In another embodiment, the nucleic acid probes may be used in direct fluorescence in situ hybridization (FISH) mapping (Trask (1991) Trends Genet. 7:149-154). Although current methods of FISH mapping favor use of large clones (several to several hundred KB; see Laan et al. (1995) Genome Res. 5:13-20), improvements in sensitivity may allow performance of FISH mapping using shorter probes.

A variety of nucleic acid amplification-based methods for genetic and physical mapping may be carried out using the nucleic acids. Examples include allele-specific amplification (Kazazian (1989) J. Lab. Clin. Med 11:95-96), polymorphism of PCR-amplified fragments (CAPS; Sheffield et al. (1993) Genomics 16:325-332), allele-specific ligation (Landegren et al. (1988) Science 241:1077-1080), nucleotide extension reactions (Sokolov (1990) Nucleic Acid Res. 18:3671), Radiation Hybrid Mapping (Walter et al. (1997) Nat. Genet. 7:22-28) and Happy Mapping (Dear and Cook (1989) Nucleic Acid Res. 17:6795-6807). For these methods, the sequence of a nucleic acid is used to design and produce primer pairs for use in the amplification reaction or in primer extension reactions. The design of such primers is well known to those skilled in the art. In methods employing PCR-based genetic mapping, it may be necessary to identify DNA sequence differences between the parents of the mapping cross in the region corresponding to the instant nucleic acid sequence. This, however, is generally not necessary for mapping methods.

Performance of the methods according to the present invention result in plants having increased yield. The trait of increased yield or increased seed yield may be combined with other economically advantageous traits, such as further yield-enhancing traits, tolerance to various stresses, traits modifying various architectural features and/or biochemical and/or physiological features.

### DESCRIPTION OF FIGURES

The present invention will now be described with reference to the following figures in which:

FIG. 1 shows a CLUSTAL W multiple alignment of several plant EFTu polypeptides. The EFTu motif is represented by [symbol] a further EFTu motif is represented by [symbol] and a GTP binding motif is represented by [symbol] .

FIG. 2 shows a binary vector for expression in Oryza sativa of a tobacco EFTu under the control of an oleosin promoter.

FIG. 3 details examples of sequences useful in performing the methods according to the present invention.

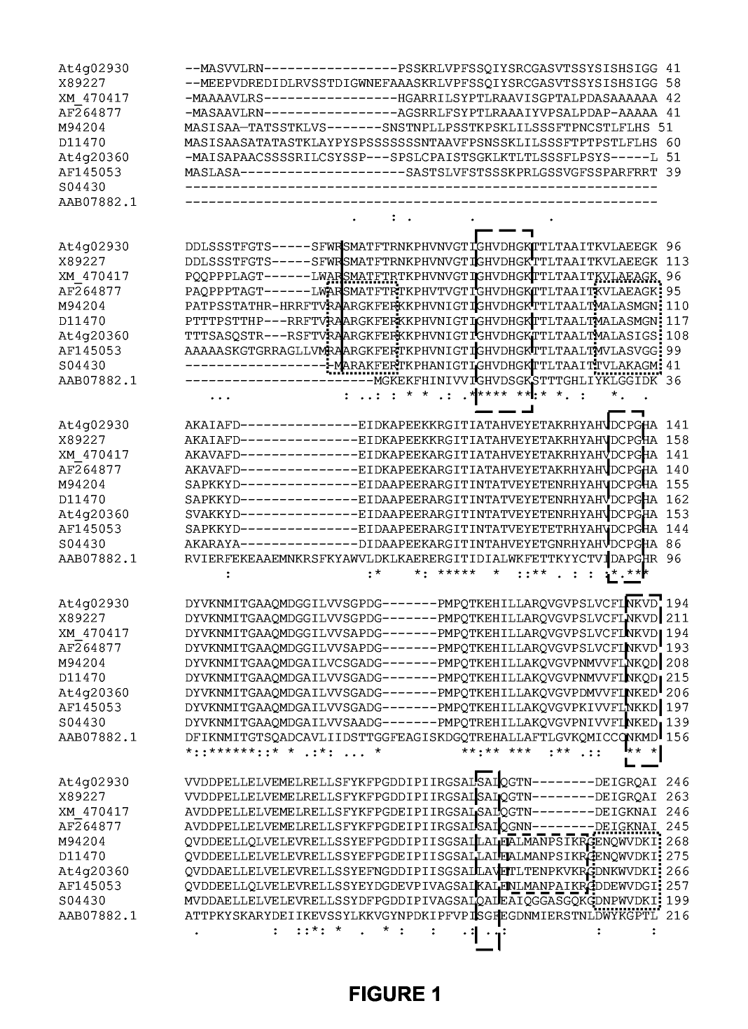

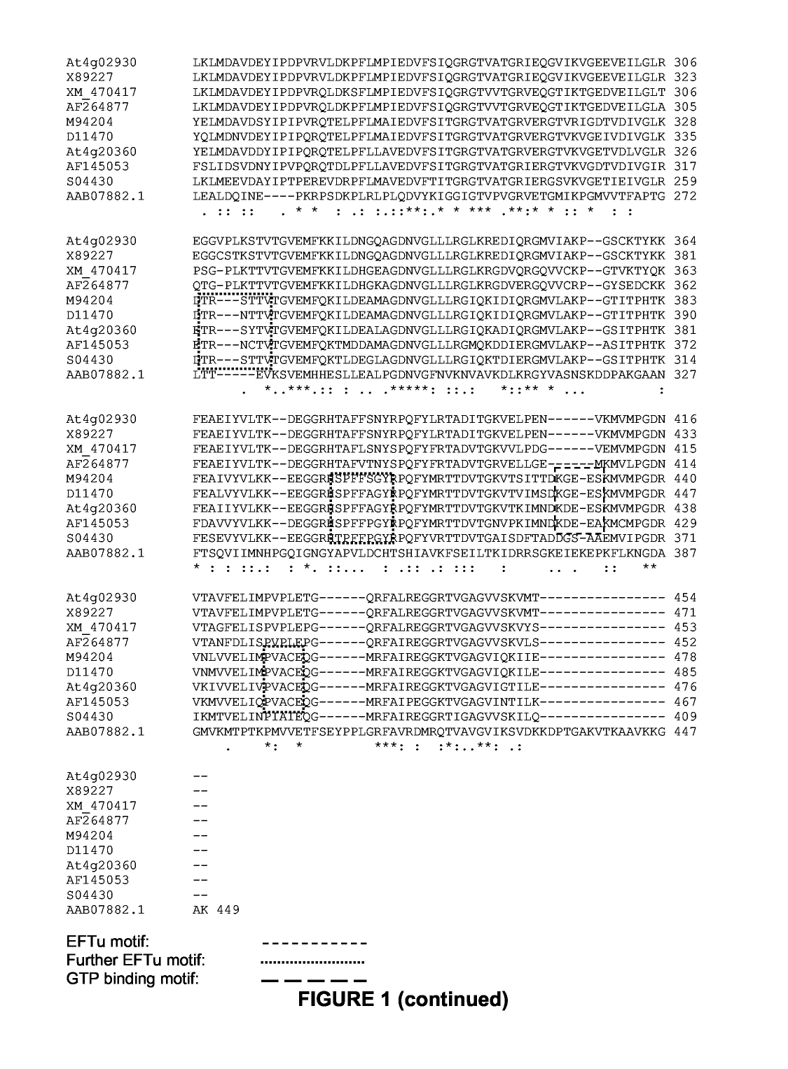

### EXAMPLES

The present invention will now be described with reference to the following examples, which are by way of illustration alone.

DNA manipulation: unless otherwise stated, recombinant DNA techniques are performed according to standard protocols described in (Sambrook (2001) Molecular Cloning: a laboratory manual, 3^{rd} Edition Cold Spring Harbor Laboratory Press, CSH, New York) or in Volumes 1 and 2 of Ausubel et al. (1994), Current Protocols in Molecular Biology, Current Protocols. Standard materials and methods for plant molecular work are described in Plant Molecular Biology Labfase (1993) by R. D. D. Croy, published by BIOS Scientific Publications Ltd (UK) and Blackwell Scientific Publications (UK).

### Example 1

### Gene Cloning

A gene encoding an EFTu protein was first identified as an expressed sequence tag from Tobacco BY2 cells and was isolated as a partial sequence in a CDNA-AFLP experiment performed with cDNA made from a synchronized tobacco BY2 cell culture (Nicotiniana tabacum L. cv. Bright Yellow-2). Based on this cDNA-AFLP experiment, BY2 tags that were cell cycle modulated were identified and selected for further cloning. The expressed sequence tags were used to screen a Tobacco cDNA library and to isolate the full length cDNA.

### Synchronization of BY2 Cells

Tobacco BY2 (Nicotiana tabacum L. cv. Bright Yellow-2) cultured cell suspension was synchronized by blocking cells in early S-phase with aphidicolin as follows. A cultured cell suspension of Nicotiana tabacum L. cv. Bright Yellow 2 was maintained as described (Nagata et al. Int. Rev. Cytol. 132, 1-30, 1992). For synchronization, a 7-day-old stationary culture was diluted 10-fold in fresh medium supplemented with aphidicolin (Sigma-Aldrich, St. Louis, Mo.; 5 mg/l), a DNA-polymerase a inhibiting drug. After 24 h, the cells were released from the block by several washings with fresh medium and they resumed their cell cycle progression.

### RNA Extraction and cDNA Synthesis

Total RNA was prepared using LiCl precipitation (Sambrook et al., 2001) and poly(A⁺) RNA was extracted from 500 μg of total RNA using Oligotex columns (Qiagen, Hilden, Germany) according to the manufacturer's instructions. Starting from 1 μg of poly(A⁺) RNA, first-strand cDNA was synthesized by reverse transcription with a biotinylated oligo-dT₂₅ primer (Genset, Paris, France) and Superscript II (Life Technologies, Gaithersburg, Md.). Second-strand synthesis was done by strand displacement with Escherichia coli ligase (Life Technologies), DNA polymerase I (USB, Cleveland, Ohio) and RNAse-H (USB).

### cDNA-AFLP Analysis

Five hundred ng of double-stranded cDNA was used for AFLP analysis as described (Vos et al., Nucleic Acids Res. 23 (21) 4407-4414, 1995; Bachem et al., Plant J. 9 (5) 745-53, 1996) with modifications. The restriction enzymes used were BstYI and MseI (Biolabs) and the digestion was done in two separate steps. After the first restriction digest with one of the enzymes, the 3′ end fragments were collected on Dyna beads (Dynal, Oslo, Norway) by means of their biotinylated tail, while the other fragments were washed away. After digestion with the second enzyme, the released restriction fragments were collected and used as templates in the subsequent AFLP steps. For pre-amplifications, a Msel primer without selective nucleotides was combined with a BstYI primer containing either a T or a C as 3′ most nucleotide. PCR conditions were as described (Vos et al., 1995). The obtained amplification mixtures were diluted 600-fold and 5 μl was used for selective amplifications using a P³³-labeled BstYI primer and the Amplitaq-Gold polymerase (Roche Diagnostics, Brussels, Belgium). Amplification products were separated on 5% polyacrylamide gels using the Sequigel system (Biorad). Dried gels were exposed to Kodak Biomax films as well as scanned in a phospholmager (Amersham Pharmacia Biotech, Little Chalfont, UK).

### Characterization of AFLP Fragments

Bands corresponding to differentially expressed transcripts, among which was the transcript corresponding to SEQ ID NO 1, were isolated from the gel and eluted DNA was reamplified under the same conditions as for selective amplification. Sequence information was obtained either by direct sequencing of the reamplified polymerase chain reaction product with the selective BstYI primer or after cloning the fragments in pGEM-T easy (Promega, Madison, Wis.) or by sequencing individual clones. The obtained sequences were compared against nucleotide and protein sequences present in the publicly available databases by BLAST sequence alignments (Altschul et al., Nucleic Acids Res. 25(17) 3389-3402 1997). When available, tag sequences were replaced with longer EST or isolated cDNA sequences to increase the chance of finding significant homology. The physical cDNA clone corresponding to SEQ ID NO 1 was subsequently amplified from a commercial Tobacco cDNA library as follows.

### Gene Cloning

A c-DNA library with average inserts of 1,400 bp was made with poly(A⁺) isolated from actively dividing, non-synchronized BY2 tobacco cells. These library-inserts were cloned in the vector pCMVSPORT6.0, comprising a attB gateway cassette (Life Technologies). From this library 46,000 clones were selected, arrayed in 384-well microtiter plates, and subsequently spotted in duplicate on nylon filters. The arrayed clones were screened by using pools of several hundreds of radioactively labeled tags as probes (among which was the BY2-tag corresponding to the sequence of SEQ ID NO 1). Positive clones were isolated (among which the clone reacting with the BY2-tag corresponding to the sequence of SEQ ID NO 1), sequenced, and aligned with the tag sequence. In cases where hybridisation with the tag failed, the full-length cDNA corresponding to the tag was selected by PCR amplification as follows. Tag-specific primers were designed using primer3 program (http://www-genome.wi.mit.edu/genome_software/other/primer3.html) and used in combination with the common vector primer to amplify partial cDNA inserts. Pools of DNA, from 50,000, 100,000, 150,000, and 300,000 cDNA clones were used as templates in PCR amplifications. Amplification products were isolated from agarose gels, cloned, sequenced and aligned with tags.

Subsequently, the full-length cDNA corresponding to SEQ ID NO 1 was cloned from the pCMVsport6.0 library vector into a suitable plant expression vector via an LR Gateway reaction.

### LR Gateway Reaction to Clone the Gene into a Plant Expression Vector

The pCMV Sport 6.0 was subsequently used in an LR reaction with a Gateway destination vector suitable for rice transformation. This vector contains as functional elements within the T-DNA borders a plant selectable marker and a Gateway cassette intended for LR in vivo recombination with the sequence of interest already cloned in the donor vector. Upstream of this Gateway cassette is the oleosin promoter for seed-specific expression of the gene.

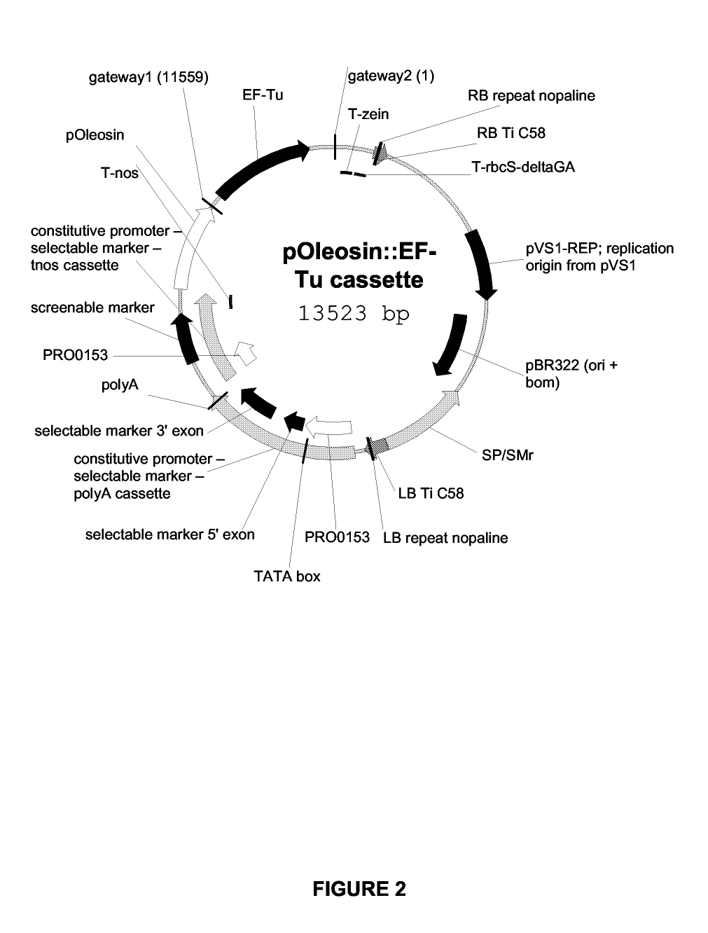

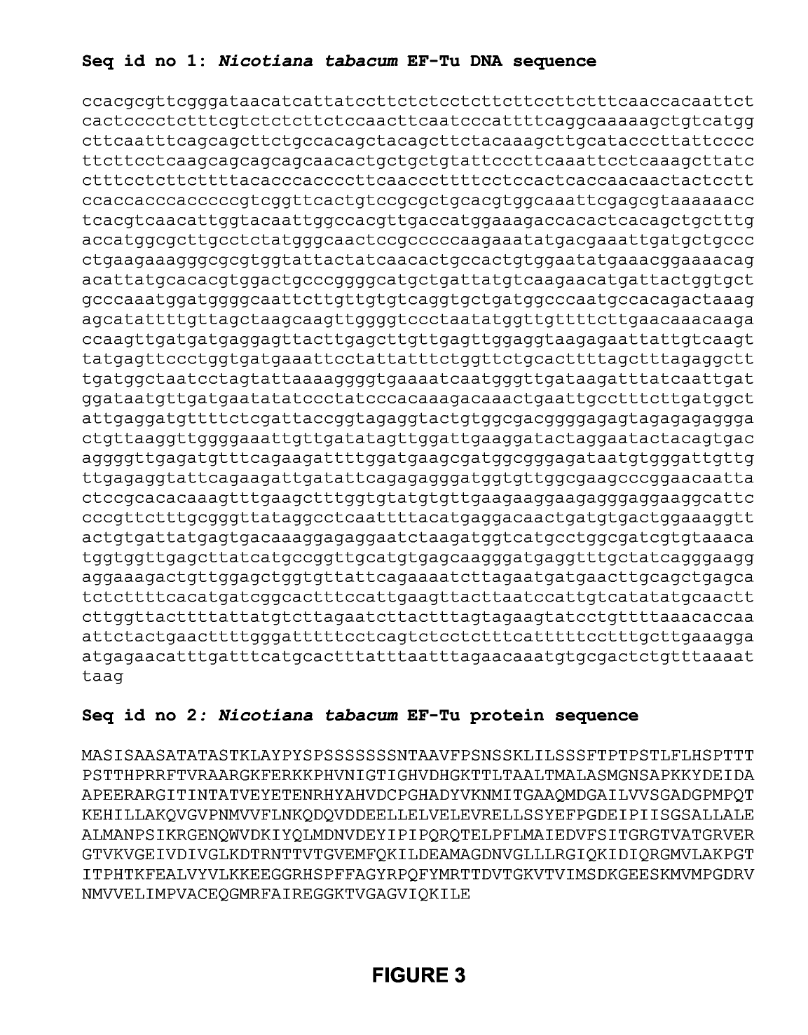

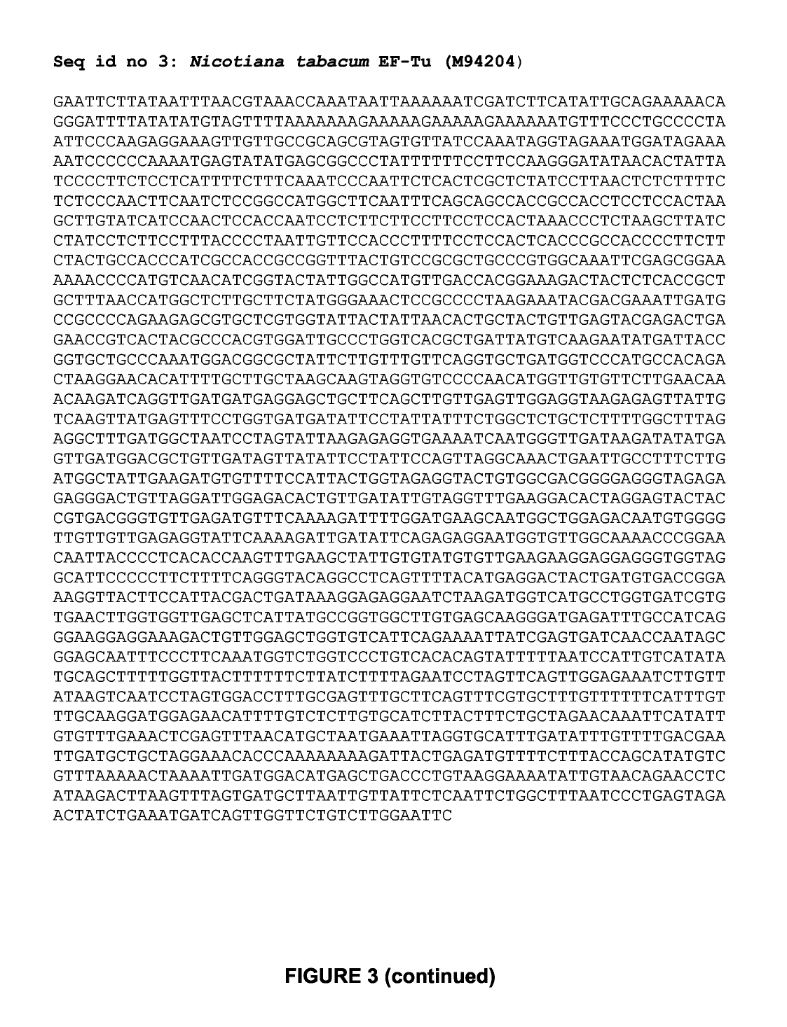

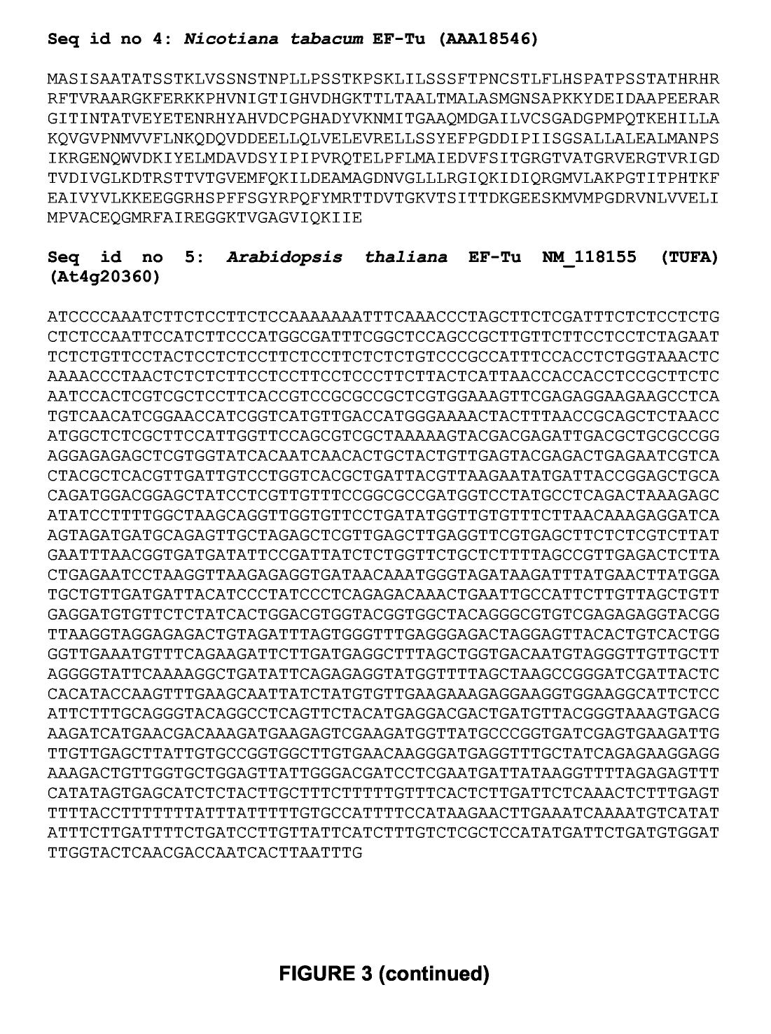

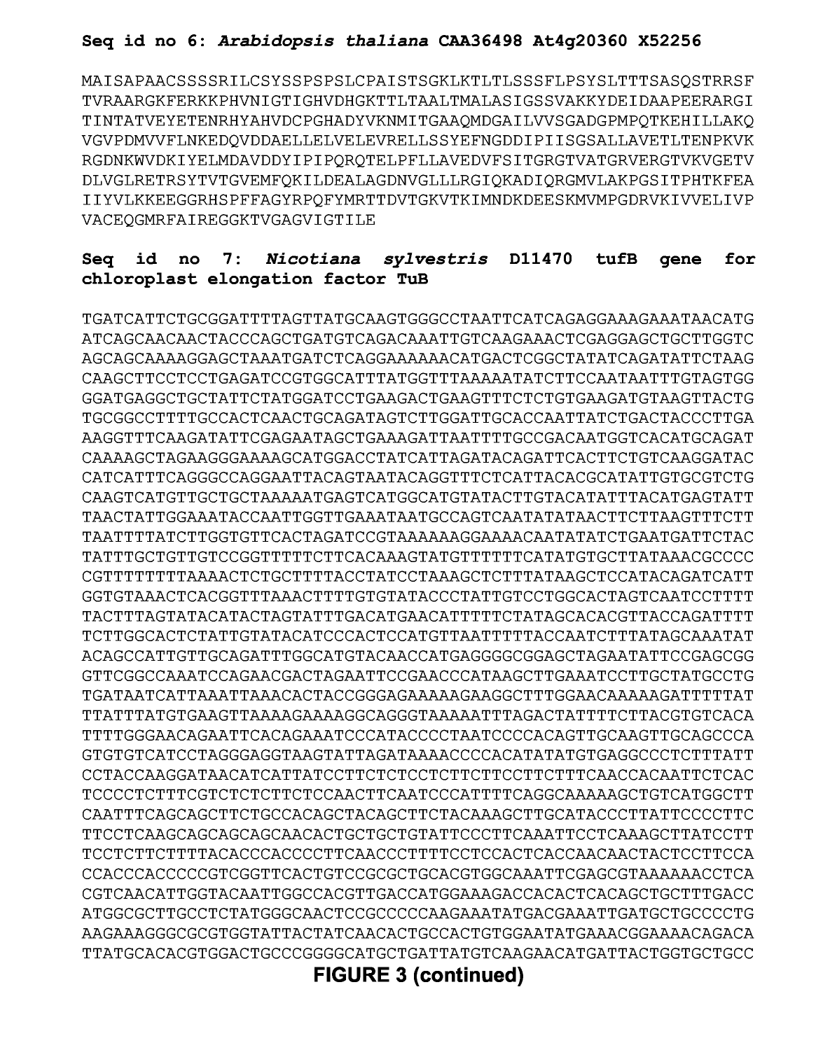

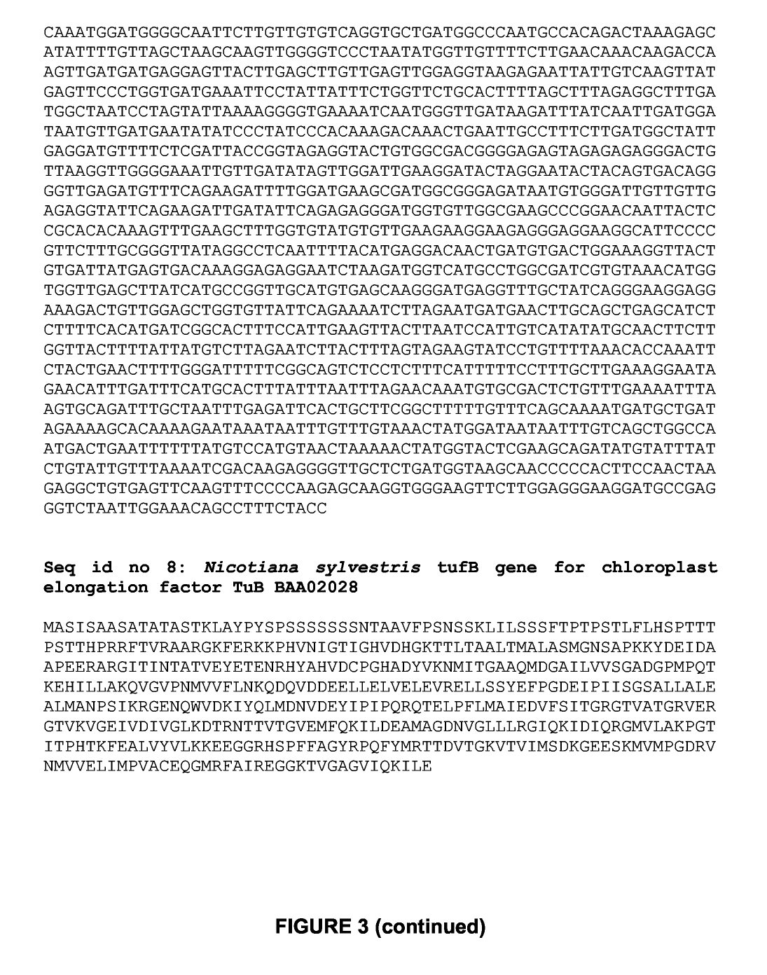

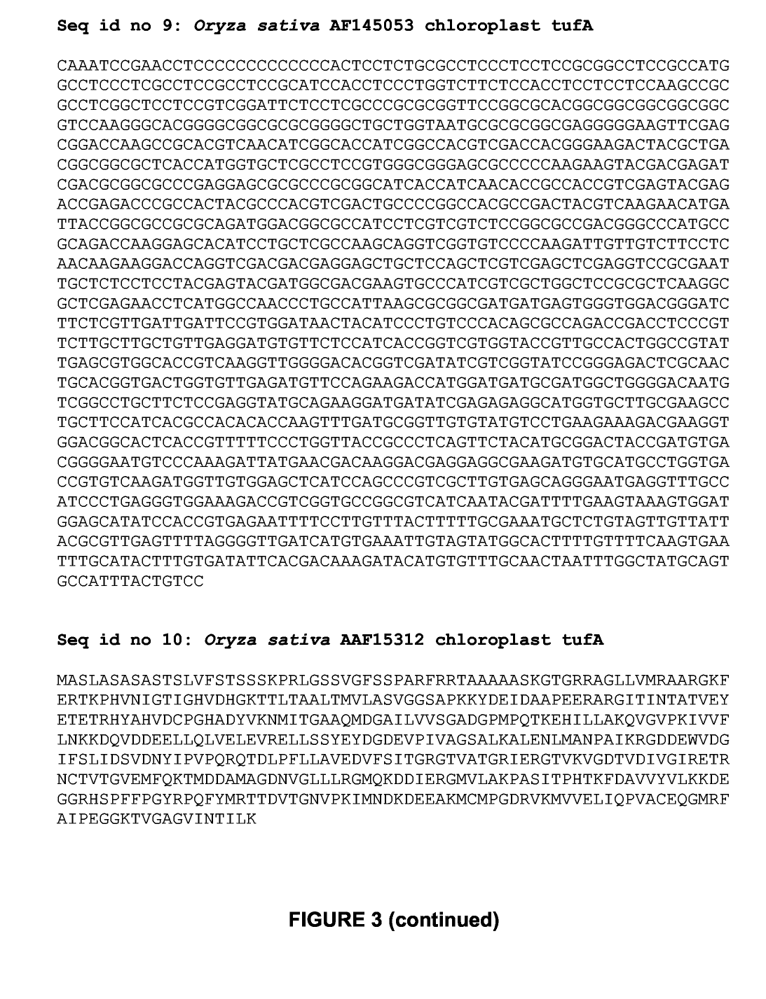

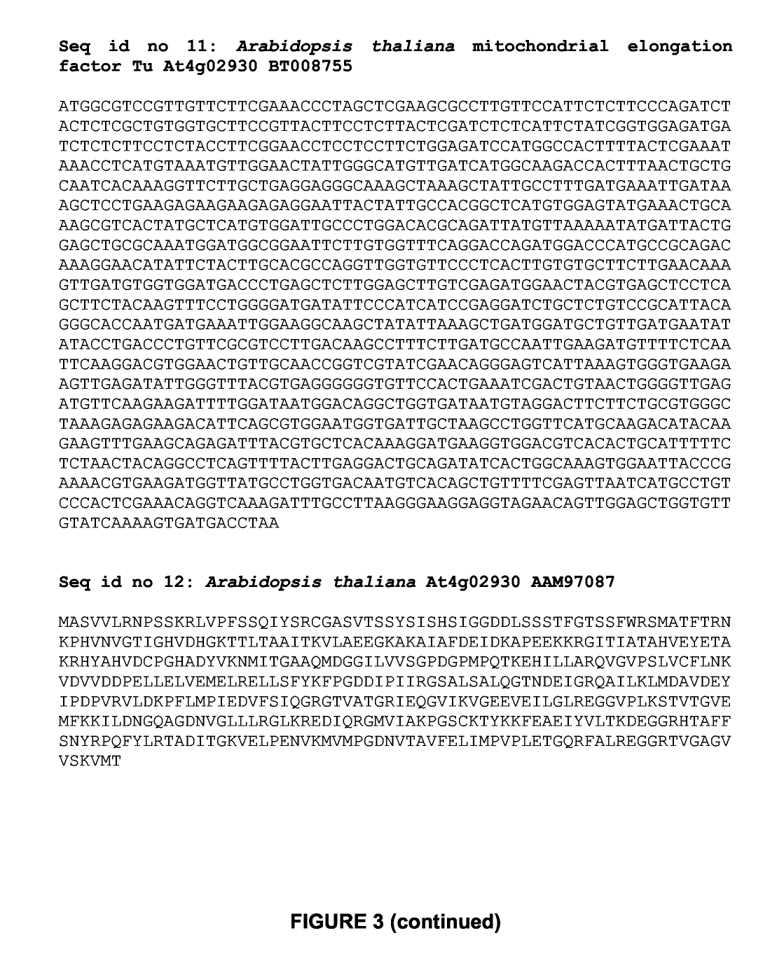

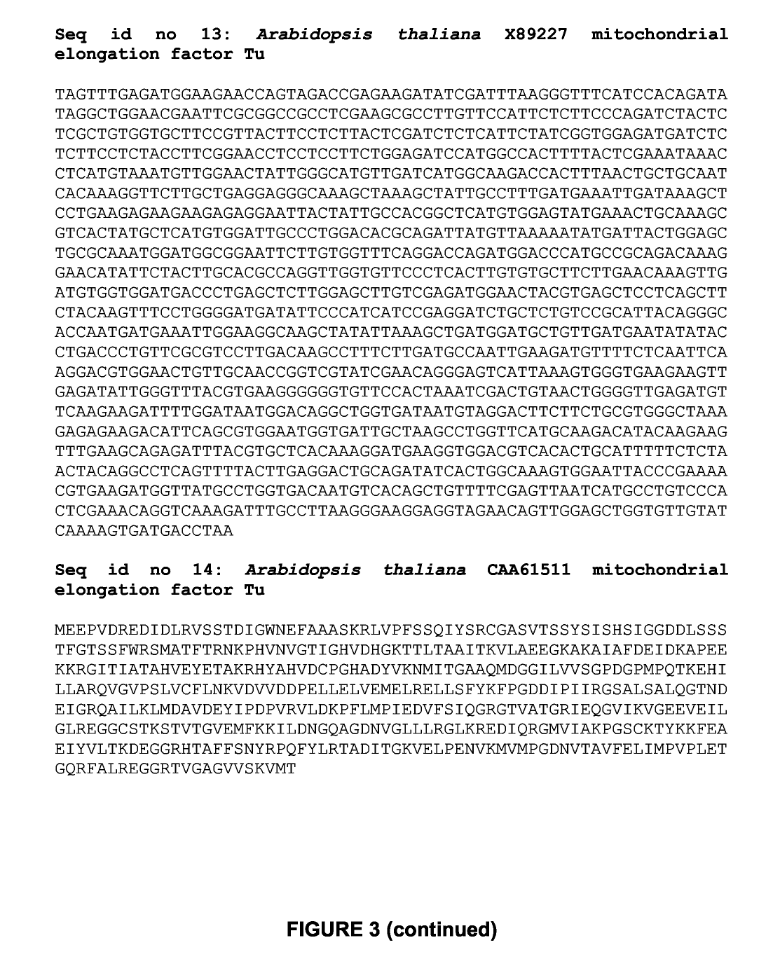

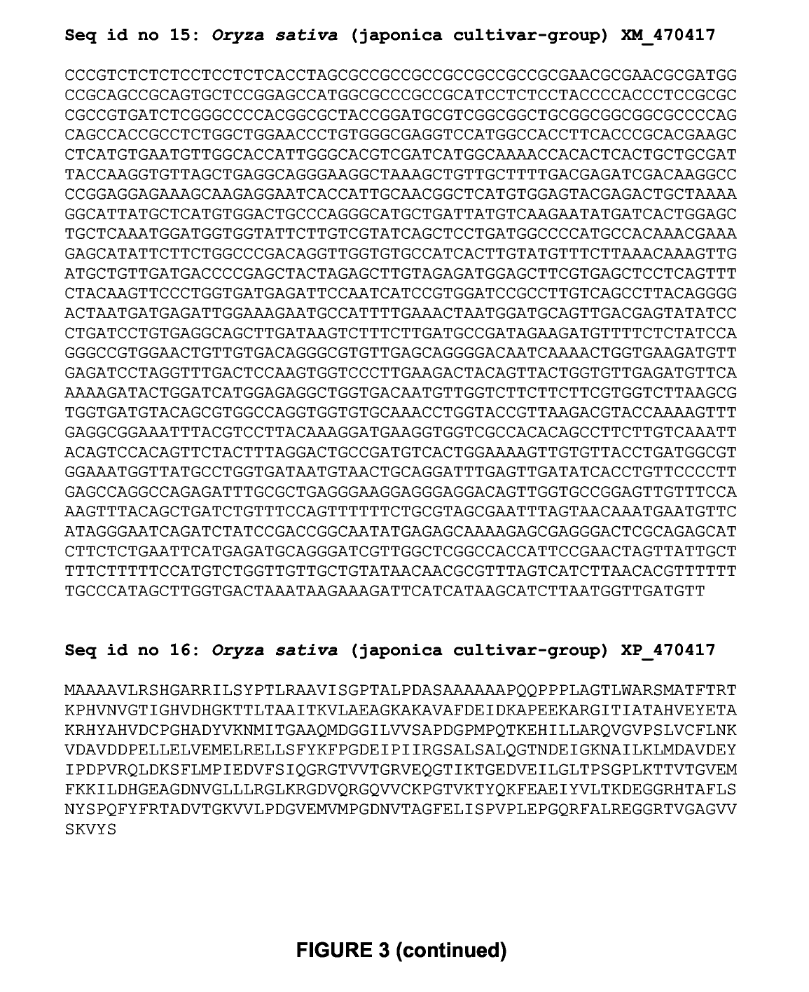

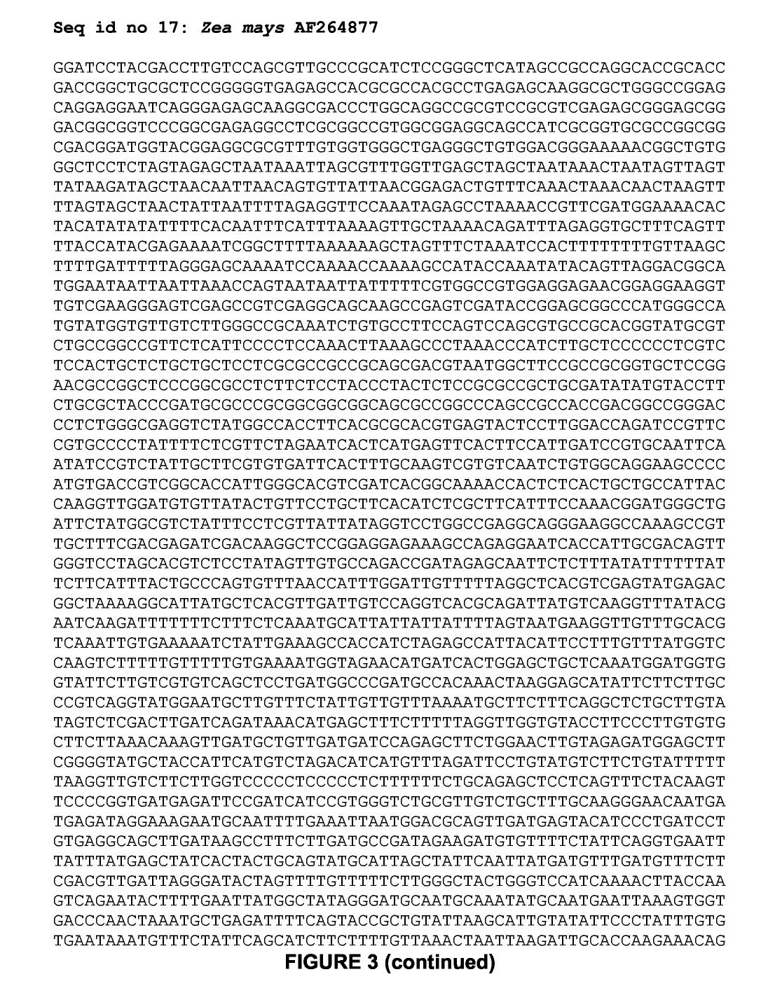

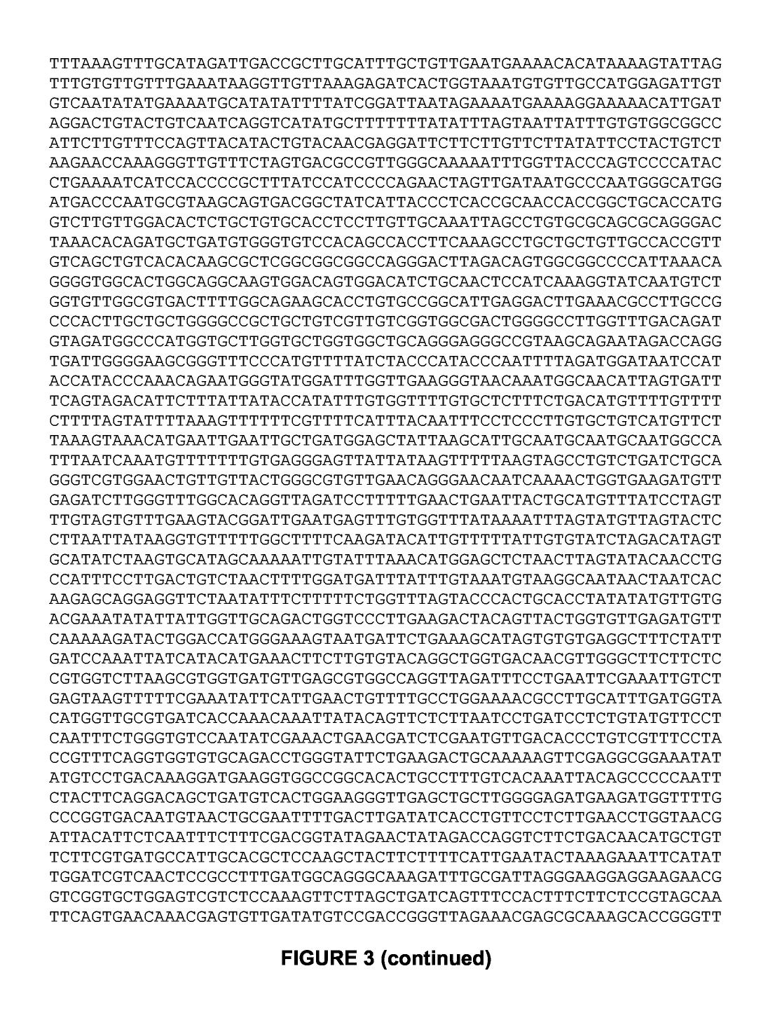

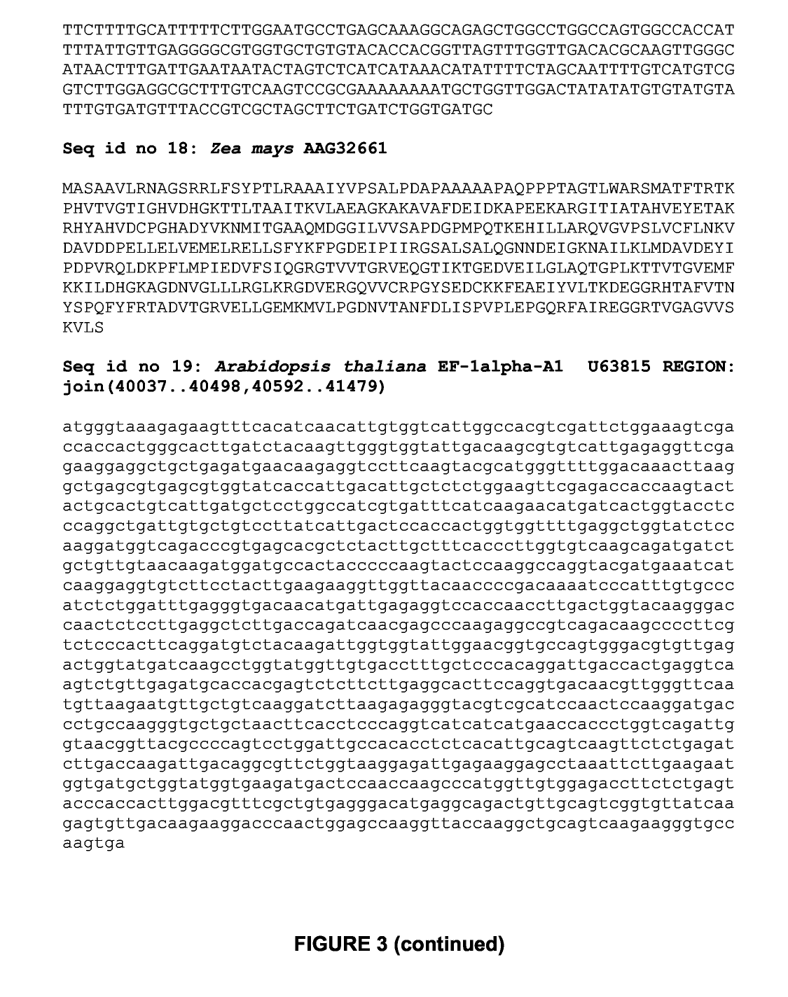

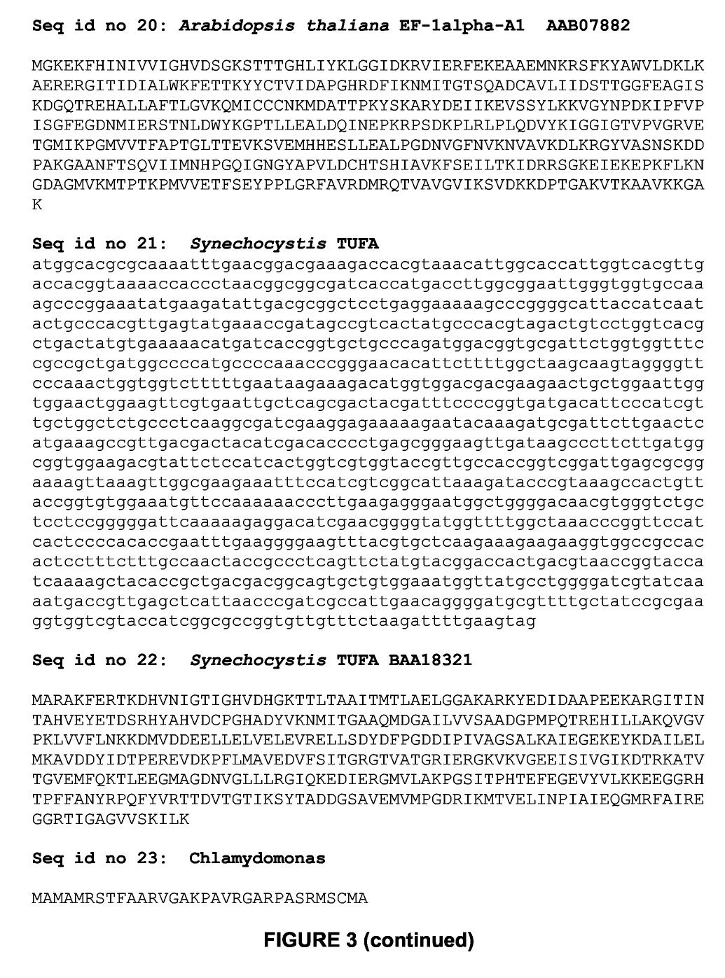

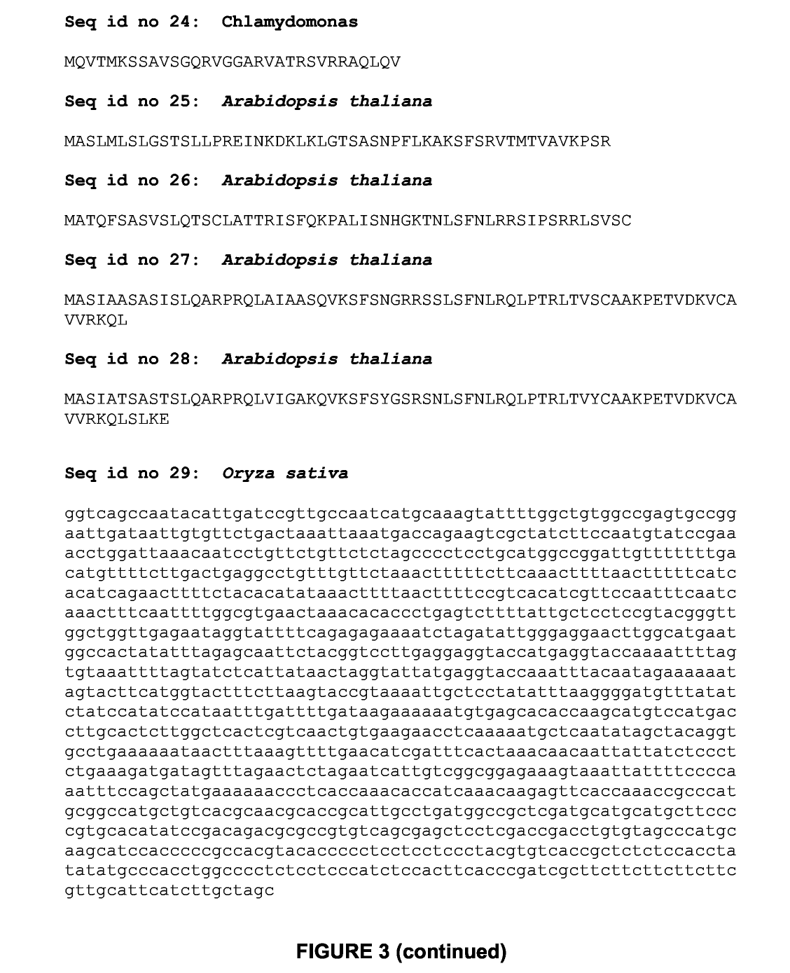

After the recombination step, the resulting expression vector (see FIG. 2) was transformed into Agrobacterium strain LBA4404 and subsequently into rice plants.

### Example 3

### Evaluation and Results

Approximately 15 to 20 independent T0 rice transformants were generated. The primary transformants were transferred from a tissue culture chamber to a greenhouse for growing and harvest of T1 seed. 5 events, of which the T1 progeny segregated 3:1 for presence/absence of the transgene, were retained. For each of these events, approximately 10 T1 seedlings containing the transgene (hetero- and homo-zygotes) and approximately 10 T1 seedlings lacking the transgene (nullizygotes) were selected by monitoring visual marker expression. T1 events were further evaluated in the T2 generation following the same evaluation procedure as for the T1 generation but with more individuals per event.

### Statistical Analysis: F-Test

A two factor ANOVA (analysis of variants) was used as a statistical model for the overall evaluation of plant phenotypic characteristics. An F-test was carried out on all the parameters measured of all the plants of all the events transformed with the gene of the present invention. The F-test was carried out to check for an effect of the gene over all the transformation events and to verify for an overall effect of the gene, also known as a global gene effect. The threshold for significance for a true global gene effect was set at a 5% probability level for the F-test. A significant F-test value points to a gene effect, meaning that it is not only the presence or position of the gene that is causing the differences in phenotype.

### 3.1 Seed-Related Parameter Measurements

The mature primary panicles were harvested, bagged, barcode-labeled and then dried for three days in an oven at 37° C. The panicles were then threshed and all the seeds were collected and counted. The filled husks were separated from the empty ones using an air-blowing device. The empty husks were discarded and the remaining fraction was counted again. The filled husks were weighed on an analytical balance. The number of filled seeds was determined by counting the number of filled husks that remained after the separation step. The total seed yield (seed weight) was measured by weighing all filled husks harvested from a plant. Total seed number per plant was measured by counting the number of husks harvested from a plant. The harvest index in the present invention is defined as the ratio of total seed yield and the above ground area (mm²) multiplied by a factor 10⁶.

### 3.2 Aboveground Area

Plant aboveground area (or area max) was determined by counting the total number of pixels from aboveground plant parts discriminated from the background. This value was averaged for the pictures taken on the same time point from the different angles and was converted to a physical surface value expressed in square mm by calibration. Experiments show that the aboveground plant area measured this way correlates with the biomass of plant parts above ground.

The Table of results below show the p values from the F test for the T1 and T2 evaluations. The percentage difference between the transgenics and the corresponding nullizygotes is also shown. For example, in the case of number of filled seeds, 2 lines in the T1 generation gave a greater than 59% difference in the number of filled seeds obtained from transgenic plants compared to the number of filled seeds obtained from corresponding nullizygotes; the p-value from the F test for these two lines was less than 0.12. Overall, 5 lines were evaluated for the number of filled seeds giving a percentage difference of 19% for the number of filled seeds of transgenics plants compared to the number of filled seeds of corresponding nullizygotes; a p value from the F test for these 5 lines gave a value of 0.05. Similarly, in the T2 generation, 1 line gave a 23% difference for the number of filled seeds obtained from transgenic plants compared to the number of filled seeds obtained from corresponding nullizygotes; the p-value from the F test for this line was 0.08. Overall, in the T2 generation, 3 lines were evaluated for the number of filled seeds giving a percentage difference between the number of filled seeds of transgenics plants compared to the number of filled seeds of corresponding nullizygotes of 11%; a p value from the F test for these 3 lines gave a value of 0.08.

| EFTu:pOleosin | | | p-value | | | p-value |
|---|---|---|---|---|---|---|
| Phenotype | T1 | % | from F | T2 | % | from the F |
| measured | Generation | Difference | test | Generation | Difference | test |
| Area max | 2 lines | >17% | <0.1 | / | / | / |
| No. filled seeds | 2 lines | >59% | <0.12 | 1 line | 23% | 0.08 |
| | overall 5 lines | 19% | 0.05 | overall 3 lines | 11% | 0.097 |
| Total Weight Seeds | 1 line | 62% | 0.091 | 1 line | 22% | 0.094 |
| | overall 5 lines | 15% | 0.15 | overall 3 lines | 11% | 0.123 |
| Harvest Index | 1 line | 46% | 0.019 | 1 line | 24% | 0.043 |
| | overall 5 lines | 16% | 0.05 | overall 3 lines | 10% | 0.1 |

1. A method for increasing plant yield under non-stress conditions, comprising increasing activity in a plastid of a translation elongation factor (EFTu) or a homologue thereof comprising: (i) the following GTP-binding domains in any order GXXXXGK and DXXG and NKXD and S/L/K/Q A/G L/V/F, wherein X is any amino acid; and (ii) EFTu domain ALMANPAIKR or a domain having at least 50% identity thereto and/or K D/G EE S/A, and selecting for plants having increased yield relative to corresponding wild type plants grown under comparable conditions.

2. The method according to claim 1, wherein said increased activity is effected by introducing a genetic modification in the locus of a gene encoding an EFTu polypeptide or a homologue thereof.

3. The method according to claim 2, wherein said genetic modification is effected by one of site-directed mutagenesis, directed evolution, homologous recombination, TILLING and T-DNA activation.

4. A method for increasing plant yield under non-stress conditions, comprising:
(i) introducing and expressing in a plant, plant part or plant cell an EFTu nucleic acid or a variant thereof encoding an EFTu polypeptide or a homologue thereof comprising: (i) the following GTP-binding domains in any order GXXXXGK and DXXG and NKXD and S/L/K/Q A/G L/V/F, wherein X is any amino acid; and (ii) EFTu domain ALMANPAIKR or a domain having at least 50% identity thereto and/or K D/G EE S/A; and
(ii) targeting the polypeptide to a plastid in the plant cell, unless the nucleic acid or variant thereof is a plastidic gene.

5. The method according to claim 4, wherein said variant is a portion of an EFTu nucleic acid or a sequence capable of hybridising to an EFTu nucleic acid, which portion or hybridising sequence encodes a polypeptide comprising: (i) the following GTP-binding domains in any order GXXXXGK and DXXG and NKXD and S/L/K/Q A/G L/V/F, wherein X is any amino acid; and (ii) EFTu domain ALMANPAIKR or a domain having at least 50% identity thereto and/or K D/G EE S/A.

6. The method according to claim 4, wherein said EFTu nucleic acid or variant thereof is overexpressed in the plant.

7. The method according to claim 4, wherein said EFTu nucleic acid or variant thereof is of plant origin.

8. The method according to claim 4, wherein said EFTu nucleic acid or variant thereof is operably linked to a seed-specific promoter.

9. The method according to claim 8, wherein said promoter is an oleosin promoter.

10. The method according to claim 1, wherein said increased yield is increased seed yield relative to corresponding wild type plants.

11. The method according to claim 1, wherein said increased yield is increased aboveground plant biomass.

12. The method according to claim 1, wherein said increased yield gives increased growth rate relative to corresponding wild type plants.

13. The method according to claim 10, wherein said increased seed yield is selected from any one or more of (i) increased seed biomass; (ii) increased number of (filled) seeds; (iii) increased seed size; (iv) increased seed volume; (v) increased harvest index; and (vi) increased thousand kernel weight (TKW).

14. A plant obtained by the method according to claim 1.

15. A construct comprising:
(i) an EFTu nucleic acid or variant thereof encoding a polypeptide comprising: (a) the following GTP-binding domains in any order GXXXXGK and DXXG and NKXD and S/L/K/Q A/G LN/F, where X is any amino acid; and (b) EFTu domain ALMANPAIKR or a domain having at least 50% identity thereto and/or K D/G EE S/A; and
(ii) one or more control sequences capable of driving expression of the nucleic acid sequence of (i); and optionally
(iii) a transcription termination sequence.

16. The construct according to claim 15, wherein said one or more control sequences comprises a seed-specific promoter.

17. The construct according to claim 16, wherein said promoter is an oleosin promoter.

18. A plant transformed with the construct according to claim 15.

19. A method for the production of a transgenic plant having increased yield under non-stressed conditions relative to yield of corresponding wild type plants grown under comparable conditions, which method comprises:
(i) introducing and expressing in a plant or plant part (including plant cell) an EFTu nucleic acid or variant thereof encoding a polypeptide comprising: (a) the following GTP-binding domains in any order GXXXXGK and DXXG and NKXD and S/L/K/Q A/G L/V/F, where X is any amino acid; and (b) EFTu domain ALMANPAIKR or a domain having at least 50% identity thereto and/or K D/G EE S/A, which polypeptide is targeted to a plastid in a plant cell if it is not already in the plastid; and
(ii) cultivating the plant or plant part under conditions promoting plant growth and development.

20. A transgenic plant grown under non-stressed conditions and having increased yield, relative to corresponding wild type plants grown under comparable conditions, said increased yield resulting from an EFTu nucleic acid or variant thereof introduced and expressed in said plant, said EFTu nucleic acid or variant thereof encoding a polypeptide comprising: (a) the following GTP-binding domains in any order GXXXXGK and DXXG and NKXD and S/L/K/Q A/G L/V/F, where X is any amino acid; and (b) EFTu domain ALMANPAIKR or a domain having at least 50% identity thereto and/or K D/G EE S/A, which polypeptide is targeted to a plastid in a plant cell if it is not already in the plastid.

21. The transgenic plant according to claim 20, wherein said plant is a monocotyledonous plant.

22. Harvestable parts of the transgenic plant according to claim 20.

23. The harvestable parts according to claim 22, wherein said harvestable parts are seeds.

24. Products derived from the transgenic plant according to claim 21 or from harvestable parts of said plant.

25. (canceled)

26. (canceled)

27. A method of selecting a plant having increased yield relative to a corresponding wild type plant, comprising utilizing an EFTu nucleic acid/gene or variant thereof or of an EFTU-like polypeptide or homologue thereof as a molecular marker.

28. The method according to claim 4, wherein said EFTu nucleic acid or variant thereof is derived from a dicotyledonous plant.

29. The method according to claim 28, wherein the dicotyledonous plant is from the family Solanaceae.

30. The method according to claim 28, wherein the dicotyledonous plant is from the genus Nicotianae.

31. The method according to claim 4, wherein said EFTu nucleic acid or variant thereof is operably linked to an embryo-specific and/or alerone-specific promoter.

32. The construct according to claim 16, wherein said seed-specific promoter is an embryo-specific and/or alerone-specific promoter.

33. The transgenic plant according to claim 20, wherein said plant is selected from the group consisting of sugarcane, rice, maize, wheat, barley, millet, rye oats or sorghum.
